(19) United States
(12) Patent Application Publication
(10) Pub. No.: US 2025/0223555 A1
(43) Pub. Date: Jul. 10, 2025

(54) REPROGRAMMING OF IMMUNE-ENHANCING NEUTROPHILS BY SUBCLINICAL LOW-DOSE ENDOTOXIN FOR THE TREATMENT OF CANCER

(71) Applicant: Virginia Tech Intellectual Properties, Inc., Blacksburg, VA (US)

(72) Inventors: Liwu Li, Blacksburg, VA (US); Yao Zhang, Blacksburg, VA (US)

(21) Appl. No.: 19/009,514

(22) Filed: Jan. 3, 2025

Related U.S. Application Data
(60) Provisional application No. 63/617,448, filed on Jan. 4, 2024.

(51) Int. Cl. C12N 5/0787; A61K 35/15; A61P 35/00

(52) U.S. Cl. CPC C12N 5/0642; A61K 35/15; A61P 35/00; C12N 2501/052; C12N 2501/22

(57) ABSTRACT

Provided are compositions and methods for reprogramming neutrophils using low-dose endotoxin for enhancing anti-tumor immune responses. The immune-enhancing neutrophils provide therapeutic applications for neutrophil-based cancer immunotherapies. Neutrophils trained by low-dose endotoxin adopt a unique immune-enhancing cluster characterized by immune-enhancing surface markers and a reduction in immune-suppressive surface markers. The immune-enhancing neutrophils exhibit relieved suppression of adaptive T cells as compared to un-trained neutrophils and enables the generation of immune-enhancing neutrophils through activating STAT5 and reducing innate suppressor IRAK-M.

### CROSS-REFERENCE TO RELATED APPLICATION(S)

This application claims the benefit under 35 U.S.C. § 119(e) to U.S. Provisional Application 63/617,448, filed Jan. 4, 2024 and entitled “REPROGRAMMING OF IMMUNE-ENHANCING NEUTROPHILS BY SUBCLINICAL LOW-DOSE ENDOTOXIN FOR THE TREATMENT OF CANCER,” which is hereby incorporated herein by reference in its entirety.

### GOVERNMENT SUPPORT CLAUSE

This invention was made with government support under R01AI136386 awarded by the National Institute of Allergy and Infectious Diseases (NIAID) of the National Institutes of Health (NIH). The government has certain rights in the invention.

### TECHNICAL FIELD

This disclosure relates to neutrophils with unique combinations of cell surface markers, capable of enhancing anti-tumor function, and more particularly to methods of isolating the neutrophils and using the neutrophils for anti-cancer treatments.

### BACKGROUND

Current anti-cancer immunotherapeutic approaches primarily focus on the adaptive immune system through tumor vaccines, engineered T cells, and check-point inhibition, however, have only demonstrate limited success. Studies have suggested that targeting adaptive immune cells alone may not be sufficient to render effective anti-cancer therapy, emphasizing the critical need to better characterize the less-explored innate immune cells. With particular relevance, innate neutrophils are the most abundant immune cells, constituting >50% of all leukocytes. The numbers of circulating neutrophils are further increased in patients with cancers including colorectal cancer (CRC), especially in patients with advanced-stage cancer. Translational studies through the last decade reveal that a higher ratio of tumor-associated neutrophils is a robust predictor of poor clinical outcomes in many solid tumors, including CRC. Recent studies reveal that neutrophils may support tumor growth through inhibiting CD8 T cells and inducing CD8 T cell apoptosis, and such inhibition can be released through applying a TGFβ inhibitor. Tumor associated neutrophils may compromise the anti-cancer immune response through expressing co-inhibitory molecules such as PD-L1 and suppressing T cell proliferation and activation.

Clinical studies further reveal that cancer patients may have heterogeneous neutrophil populations with both immune-enhancing (N1) and immune-inhibiting (N2) phenotypes. Consequently, attempts to deplete immune-suppressive neutrophils have been shown to have beneficial effects in reducing tumor progression. A recent clinical and translational study revealed that, instead of the engineered CAR-T cells, neutrophils are predominantly responsible for the broad-spectrum eradication of heterogeneous tumor cells in vivo. Together, these studies point to an intriguing potential of re-programming neutrophils in cancer treatment. Despite its compelling prognostic value, the mechanisms underlying the tumor-promoting or inhibiting activity of neutrophils remain poorly understood.

Neutrophils are not only known to be closely associated with the pathogenesis of cancers, such as intestinal cancer, but may also be utilized to treat the cancer if properly reprogrammed. However, in order to better harness the significant potential of neutrophils in anti-cancer therapy, an improved understanding of neutrophil reprogramming dynamics responsible for the immune-inhibiting vs immune-enhancing effects on the tumor immune environments is needed.

Therefore, there remains a need for novel neutrophil therapies and methods of isolating such neutrophil therapies for use in treating cancer.

### SUMMARY

In Example 1, a method of reprogramming neutrophils into immune-enhancing neutrophils comprises receiving innate neutrophils isolated from a heterologous or allogenic donor, and priming the innate neutrophils with a low-dose endotoxin to result in immune-enhancing neutrophils, wherein the immune-enhancing neutrophils express surface markers comprising CD177^{lo} and at least one of Dectin2 (Clec4n)^{hi} or EHD1^{hi}.

Example 2 relates to the method according to Example 1, wherein the low-dose endotoxin comprises lipopolysaccharide (LPS).

Example 3 relates to the method according to Example 1, wherein the low-dose endotoxin is provided at a concentration of between about 10 pg/mL and about 1000 pg/mL.

Example 4 relates to the method according to Example 1, wherein the surface markers comprise CD177^{lo} and Dectin 2 (Clec4n)^{hi}, or wherein the surface markers comprise CD177^{lo} and EHD1^{hi}.

Example 5 relates to the method according to Example 1, wherein the surface markers comprise CD177^{lo}, Dectin2 (Clec4n)^{hi}, and EHD1^{hi}.

Example 6 relates to the method according to Example 1, wherein the immune-enhancing neutrophils exhibit elevated expression of immune-enhancing genes comprising CD44, CD80, CD86, CD74, EHD1, Dectin2, CD40, CD200R, CD62L, or a combination thereof.

Example 7 relates to the method according to Example 6, wherein the immune-enhancing neutrophils exhibit a reduced expression of CD11b.

Example 8 relates to the method according to Example 1, wherein the priming of the innate neutrophils with the low-dose endotoxin result in a reduction of IRAK-M in the immune-enhancing neutrophils.

Example 9 relates to the method according to Example 1, further comprising a step of isolating the immune-enhancing neutrophils expressing surface markers comprising CD177^{lo} and at least one of Dectin2 (Clec4n)^{hi} or EHD1^{hi} by flow cytometry or magnetic bead-based separation.

Example 10 relates to the method according to Example 1, wherein the innate neutrophils are isolated from human peripheral blood, and wherein the innate neutrophils are further treated with low dose granulocyte-macrophage colony-stimulating factor (GM-CSF) or granulocyte colony-stimulating factor (G-CSF).

Example 11 relates to the method according to Example 10, wherein the concentration of low dose GM-CSF or G-CSF is in a range of from about 0.1 ng/mL to about 5 ng/mL.

In Example 12, a method of treating cancer in a subject comprises receiving innate neutrophils isolated from a heterologous or allogenic donor, priming the innate neutrophils with a low-dose endotoxin to result in immune-enhancing neutrophils, wherein the immune-enhancing neutrophils express surface markers comprising CD177^{lo} and at least one of Dectin2 (Clec4n)^{hi} or EHD1^{hi}, and administering the immune-enhancing neutrophils to the subject.

Example 13 relates to the method according to Example 12, wherein the innate neutrophils are isolated from human peripheral blood, and wherein the innate neutrophils are further treated with low dose granulocyte-macrophage colony-stimulating factor (GM-CSF) or granulocyte colony-stimulating factor (G-CSF).

Example 14 relates to the method according to Example 12, wherein the immune-enhancing neutrophils are administered to the subject at least once per month or at least once weekly.

Example 15 relates to the method according to Example 12, wherein the immune-enhancing neutrophils are administered to the subject in the form of a blood transfusion via an intravenous route of administration.

Example 16 relates to the method according to Example 15, wherein between about 50 mL and about 500 mL of blood containing the immune-enhancing neutrophils is administered to the subject via the blood transfusion.

Example 17 relates to the method according to Example 12, wherein the low-dose endotoxin comprises lipopolysaccharide (LPS), and wherein the LPS is provided at a concentration of between about 10 pg/mL and about 1000 pg/mL.

Example 18 relates to the method according to Example 12, wherein the surface markers comprise (i) CD177^{lo} and Dectin 2 (Clec4n)^{hi}; (ii) CD177^{lo} and EHD1^{hi}; or (iii) CD177^{lo}, Dectin2 (Clec4n)^{hi}, and EHD1^{hi}.

Example 19 relates to the method according to Example 12, wherein the immune-enhancing neutrophils exhibit elevated expression of immune-enhancing genes comprising CD44, CD80, CD86, CD74, EHD1, Dectin2, CD40, CD200R, CD62L, or a combination thereof, and wherein the immune-enhancing neutrophils exhibit a reduced expression of CD11b.

Example 20 relates to the method according to Example 12, wherein the cancer is at least one of colon, lung, breast, bladder, prostate cancer, or other solid tumor cancer.

While multiple embodiments are disclosed, still other embodiments of the present disclosure will become apparent to those skilled in the art from the following detailed description, which shows and describes illustrative embodiments of the disclosure. Accordingly, the figures and detailed description are to be regarded as illustrative in nature and not restrictive.

### BRIEF DESCRIPTION OF DRAWINGS

FIG. 1 shows graphs of bone marrow neutrophils treated with high dose of LPS (100 ng/ml) or PBS as control overnight, then subjected to flow cytometry. CD11b, PD-L1, and CD62L expression on CD11b+Ly6G+neutrophils were analyzed with n≥4. Significance was calculated by unpaired two-tailed student t test. The error bars represent means±SD.

FIG. 2 shows a UMAP diagram of scRNA-Seq data from purified neutrophils treated with 100 pg/ml LPS or PBS as a control. Control neutrophils cluster into three clusters: CD177high (CD177^{hi}), CD177intermediate (CD177^{im}), and CD177low (CD177^{lo}).

FIG. 3 shows a dot plots of relative gene expression levels in different clusters of neutrophils.

FIG. 4 shows heatmaps of representative genes in purified neutrophils challenged with super-low dose of LPS (100 pg/ml).

FIG. 5 shows graphs of bone marrow neutrophils treated with 100 pg/ml of LPS or PBS as control overnight, then subjected to flow cytometry analysis. Mean fluorescence intensity (MFI) of selected proteins on CD11b+Ly6G+ neutrophils were analyzed with n≥3. Data are represented as means±SD. Significance was calculated by unpaired two-tailed student t test. *p<0.05; **p<0.01; ***p<0.001.

FIG. 6 shows graphs of bone marrow neutrophils treated with super-low dose of LPS (100 pg/ml) or PBS as control overnight, then subjected to flow cytometry. CD62L, PD-L1, Dectin1, and CD200R expression on CD11b+Ly6G+ neutrophils were analyzed at n≥3. Data are represented as means±SD. Significance was calculated by unpaired two-tailed student t test. *p<0.05; ***p<0.001.

FIG. 7 shows a schematic experimental design for the adoptive transfer (A.T.) of neutrophils in the AOM-DSS-induced colitis associated colon cancer model. AOM-DSS treated WT mice were given with LPS or PBS primed neutrophils via intravenous injection (A.T.).

FIG. 8 shows representative images of colons from the mice receiving PBS or LPS primed neutrophils during AOM-DSS treatment. Colon tumor counts (n=5). Diameter of tumors greater than or equal to 2 mm were defined as “macro” tumor, and a diameter of tumors less than 2 mm were defined as “micro” tumor. Data are represented as means±SD. Significance was calculated by two-way ANOVA with Sidak post hoc test. **p<0.01.

FIG. 9A shows a graph of body weight changes during AOM-DSS treatment. FIG. 9B shows a graph of stool clinical scores including stool consistency and bleeding. n=5. Data are represented as means±SD. Significance was calculated by two-way ANOVA with Sidak post hoc test. **p<0.01; ***p<0.001.

FIG. 10 shows graphs of the percentages of neutrophils in various tissues from AOM-DSS challenged mice with adoptive transfer of neutrophils analyzed by flow cytometry. n≥5 per group. Data are represented as means±SD. Significance was calculated by unpaired two-tailed student t test. *p<0.05.

FIG. 11 shows graphs of cell counts of CD4+ and CD8+ T cells in the spleen in mice receiving LPS-primed neutrophils compared with mice receiving PBS-primed neutrophils during AOM-DSS treatment. n≥5. Data are represented as means±SD. Significance was calculated by unpaired two-tailed student t test. *p<0.05; **p<0.01.

FIG. 12 shows graphs of percentages of CD4+ and CD8+ T cells in the mesenteric lymph nodes in mice receiving LPS-primed neutrophils compared with mice receiving PBS-primed neutrophils during AOM-DSS treatment. n≥5. Data are represented as means±SD. Significance was calculated by unpaired two-tailed student t test. *p<0.05; **p<0.01.

FIG. 13A shows graphs of the percentages of CD4+ T cells in the colon and PD-1 expression on CD4+ T cells. FIG. 13B shows graphs of the percentages of CD8+ T cells in colon and pD-1 expression on CD8+ T cells. FIG. 13C shows graphs of the percentages NK cells in colon and CD107a expression on NK cells. FIG. 13D shows graphs of Granzyme B and IFNγ levels in colon lysate, normalized on protein weight. n≥5. Data are represented as means±SD. Significance was calculated by unpaired two-tailed student t test. *p<0.05; **p<0.01.

FIG. 14 shows graphs of Ki67 expression in splenic T cells in mice receiving LPS-primed neutrophils compared with mice receiving PBS-primed neutrophils during AOM-DSS treatment. n≥5. Data are represented as means±SD. Significance was calculated by unpaired two-tailed student t test. *p<0.05; **p<0.01.

FIG. 15 shows graphs of Foxp3 expression in splenic CD4+ T cells and CD122 expression in splenic CD8+ T cells in mice receiving LPS-primed neutrophils compared with mice receiving PBS-primed neutrophils during AOM-DSS treatment. n≥5. Data are represented as means±SD. Significance was calculated by unpaired two-tailed student t test. *p<0.05; **p<0.01.

FIG. 16 shows graphs of Foxp3 expression in CD4+ T cells and CD122 expression in CD8+ T cells in mice receiving LPS-primed neutrophils compared with mice receiving PBS-primed neutrophils during AOM-DSS treatment. n≥5. Data are represented as means±SD. Significance was calculated by unpaired two-tailed student t test. *p<0.05.

FIG. 17 shows a graph of CD62L expression in splenic CD4+ cells in mice receiving LPS-primed neutrophils compared with mice receiving PBS-primed neutrophils during AOM-DSS treatment. n≥5. Data are represented as means±SD. Significance was calculated by unpaired two-tailed student t test. *p<0.05; **p<0.01.

FIG. 18 shows a graph of CD107a expression in splenic CD8+ T cells in mice receiving LPS-primed neutrophils compared with mice receiving PBS-primed neutrophils during AOM-DSS treatment. n≥5. Data are represented as means±SD. Significance was calculated by unpaired two-tailed student t test. *p<0.05; **p<0.01.

FIG. 19 shows graphs of splenocytes lymphocytes isolated from the experimental mice were cultured in RPMI completed medium supplemented with golgi blocker for 4 hours. Then IFNγ and Granzyme B (GrzB) expression were examined via flow cytometry in mice receiving LPS-primed neutrophils compared with mice receiving PBS-primed neutrophils during AOM-DSS treatment. n≥5. Data are represented as means±SD. Significance was calculated by unpaired two-tailed student t test. *p<0.05; **p<0.01.

FIG. 20 shows graphs of CD107a and Granzyme B (GrzB) levels in CD8+ T cells in mice receiving LPS-primed neutrophils compared with mice receiving PBS-primed neutrophils during AOM-DSS treatment. n≥5. Data are represented as means±SD. Significance was calculated by unpaired two-tailed student t test. *p<0.05.

FIG. 21 shows graphs of PD-1 and Tim3 expression in splenic CD4+ and CD8+ T cells in mice receiving LPS-primed neutrophils compared with mice receiving PBS-primed neutrophils during AOM-DSS treatment. n≥5. Data are represented as means±SD. Significance was calculated by unpaired two-tailed student t test. *p<0.05; **p<0.01.

FIG. 22 shows graphs of PD-1 and Tim3 expression in CD4+ and CD8+ T cells. n≥5. Data are represented as means±SD. Significance was calculated by unpaired two-tailed student t test. *p<0.05.

FIG. 23A shows a graph of cell counts of NK cells in the spleen in mice receiving LPS-primed neutrophils compared with mice receiving PBS-primed neutrophils during AOM-DSS treatment. FIG. 23B shows a graph of the percentages of NK cells in the mesenteric lymph nodes in mice receiving LPS-primed neutrophils compared with mice receiving PBS-primed neutrophils during AOM-DSS treatment. FIG. 23C shows a graph of NKG2A expression in NK cells of the spleen in mice receiving LPS-primed neutrophils compared with mice receiving PBS-primed neutrophils during AOM-DSS treatment. FIG. 23D shows NKG2A expression in NK cells of the mesenteric lymph nodes in mice receiving LPS-primed neutrophils compared with mice receiving PBS-primed neutrophils during AOM-DSS treatment. FIG. 23E shows graphs of CD107a and granzyme B expression in NK cells of the spleen in mice receiving LPS-primed neutrophils compared with mice receiving PBS-primed neutrophils during AOM-DSS treatment. FIG. 23F shows CD107a and granzyme B expression in NK cells of the mesenteric lymph nodes in mice receiving LPS-primed neutrophils compared with mice receiving PBS-primed neutrophils during AOM-DSS treatment. n≥5. Data are represented as means±SD. Significance was calculated by unpaired two-tailed student t test. *p<0.05; **p<0.01; ***p<0.001.

FIG. 24 shows a graph of CFSE-labeled T cells cocultured with LPS-primed neutrophils for 72 hours. Representative results of CFSE signal gated on CD4+ or CD8+ T cells are shown.

FIG. 25 shows graphs of the quantification of the percentages of proliferation in CD4+ or CD8+ T cells in mice receiving LPS-primed neutrophils compared with mice receiving PBS-primed neutrophils during AOM-DSS treatment. n=3. Data are represented as means±SD. Significance was calculated by unpaired two-tailed student t test. *p<0.05; **p<0.01.

FIG. 26 shows a representative dot plot of CD62L expression on CD4+ or CD8+ T cells after three days coculture.

FIG. 27 shows graphs of the quantification of CD62L^{low} percentages in CD4+ or CD8+ T cells in mice receiving LPS-primed neutrophils compared with mice receiving PBS-primed neutrophils during AOM-DSS treatment. n=5. Data are represented as means±SD. Significance was calculated by unpaired two-tailed student t test. *p<0.05; **p<0.01.

FIG. 28 shows graphs of CD107a, CD69 and PD-L1 expression in CD8+ T cells after three days coculture in mice receiving LPS-primed neutrophils compared with mice receiving PBS-primed neutrophils during AOM-DSS treatment. n=3. Data are represented as means±SD. Significance was calculated by unpaired two-tailed student t test. *p<0.05; **p<0.01.

FIG. 29 shows graphs of IFNγ and Granzyme B levels in conditioned medium in mice receiving LPS-primed neutrophils compared with mice receiving PBS-primed neutrophils during AOM-DSS treatment. n=3. Data are represented as means±SD. Significance was calculated by unpaired two-tailed student t test. *p<0.05; **p<0.01.

FIG. 30 shows graphs of CFSE-labeled NK cells cocultured with LPS-primed neutrophils for four days. Representative results of CFSE signal gated on NK1.1+CD3− NK cells are shown.

FIG. 31 shows a graph of the quantification of the percentages of proliferation in NK cells in mice receiving LPS-primed neutrophils compared with mice receiving PBS-primed neutrophils. n=5. Data are represented as means±SD. Significance was calculated by unpaired two-tailed student t test. *p<0.05.

FIG. 32 shows a graph of NKG2A expression in NK cells after coculture in mice receiving LPS-primed neutrophils compared with mice receiving PBS-primed neutrophils. n=3. Data are represented as means±SD. Significance was calculated by unpaired two-tailed student t test. *p<0.05.

FIG. 33 shows graphs of IFNγ and Granzyme B levels in conditioned medium in mice receiving LPS-primed neutrophils compared with mice receiving PBS-primed neutrophils. n=3. Data are represented as means±SD. Significance was calculated by unpaired two-tailed student t test. *p<0.05.

FIG. 34 shows a graph of LDH levels in conditioned medium from NK cell killing assay in mice receiving LPS-primed neutrophils compared with mice receiving PBS-primed neutrophils. n=3. Data are represented as means±SD. Significance was calculated by unpaired two-tailed student t test. *p<0.05.

FIG. 35 shows a graph of killing rates percent in mice receiving LPS-primed neutrophils compared with mice receiving PBS-primed neutrophils as determined by counting remaining live target cells. YAC-1 cells were used as target cells to evaluate NK cytotoxicity. n=4. Data are represented as means±SD. Significance was calculated by unpaired two-tailed student t test. *p<0.05.

FIG. 36 shows protein levels of pSTAT5 and STAT5, and relative expressions normalized to 3-actin in neutrophils treated with super-low dose LPS compared to PBS-primed neutrophils. n=4. Data are represented as means±SD. Significance was calculated by unpaired two-tailed student t test. *p<0.05; **p<0.01; ***p<0.001.

FIG. 37 shows protein levels and relative expressions of PSMD10 in neutrophils treated with super-low dose LPS compared to PBS-primed neutrophils. n=4. Data are represented as means±SD. Significance was calculated by unpaired two-tailed student t test. *p<0.05; **p<0.01; ***p<0.001.

FIG. 38 shows protein levels and relative expressions of IRAK-M in LPS-primed neutrophils compared to PBS-primed neutrophils. n=3. Data are represented as means±SD. Significance was calculated by unpaired two-tailed student t test. *p<0.05; **p<0.01; ***p<0.001.

FIG. 39 shows protein levels and relative expressions of KLF4 in neutrophils treated with super-low dose LPS compared to PBS-primed neutrophils. n=4. Data are represented as means±SD. Significance was calculated by unpaired two-tailed student t test. *p<0.05; **p<0.01; ***p<0.001.

Various embodiments of the present disclosure will be described in detail with reference to the figures. Reference to various embodiments does not limit the scope of the disclosure. Figures represented herein are not limitations to the various embodiments according to the disclosure and are presented for exemplary illustration of the disclosure.

### DETAILED DESCRIPTION

The embodiments of this disclosure are not limited to particular neutrophil compositions and methods of use or isolation, which can vary and are understood by skilled artisans. It is further to be understood that all terminology used herein is for the purpose of describing particular embodiments only and is not intended to be limiting in any manner or scope. So that the present disclosure may be more readily understood, certain terms are first defined. Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments of the present disclosure without undue experimentation, the preferred materials and methods are described herein. In describing and claiming the embodiments of the present disclosure, the following terminology will be used in accordance with the definitions set out below.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾. This applies regardless of the breadth of the range.

The term “about,” as used herein, refers to variation in the numerical quantity that can occur, for example, through typical measuring techniques and equipment, with respect to any quantifiable variable, including, but not limited to, mass, volume, temperature, and time. Further, given solid and liquid handling procedures used in the real world, there is certain inadvertent error and variation that is likely through differences in the manufacture, source, or purity of the ingredients used to make the compositions or carry out the methods and the like. Whether or not modified by the term “about,” the claims include equivalents to the quantities.

The term “actives” or “percent actives” or “percent by weight actives” or “actives concentration” are used interchangeably herein and refers to the concentration of those ingredients involved in cleaning expressed as a percentage minus inert ingredients such as water or salts. It is also sometimes indicated by a percentage in parentheses, for example, “chemical (10%).”

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques.

As used herein, “agent” can refer to any substance, compound, molecule, and the like, which can be biologically active or otherwise can induce a biological and/or physiological effect on a subject to which it is administered to. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

As used herein, the term “cancer” refers to cells having the capacity for autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, cancer of the small intestine, and cancer of the esophagus. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. In some embodiments, the present methods can be used to treat a subject having an epithelial cancer, e.g., a solid tumor of epithelial origin, e.g., lung, breast, ovarian, prostate, renal, pancreatic, or colon cancer.

As used herein, “control” can refer to an alternative subject or sample used in an experiment for comparison purpose and included to minimize or distinguish the effect of variables other than an independent variable. A “suitable control” is one that will be instantly appreciated by one of ordinary skill in the art as one that is included such that it can be determined if the variable being evaluated an effect, such as a desired effect or hypothesized effect. One of ordinary skill in the art will also instantly appreciate based on inter alia, the context, the variable(s), the desired or hypothesized effect, what is a suitable or an appropriate control needed.

As used herein, “deoxyribonucleic acid (DNA)” and “ribonucleic acid (RNA)” generally refers to any polyribonucleotide or polydeoxribonucleotide, which may be unmodified RNA or DNA or modified RNA or DNA. RNA can be in the form of non-coding RNA such as tRNA (transfer RNA), snRNA (small nuclear RNA), rRNA (ribosomal RNA), anti-sense RNA, RNAi (RNA interference construct), siRNA (short interfering RNA), microRNA (miRNA), or ribozymes, aptamers, guide RNA (gRNA) or coding mRNA (messenger RNA).

As used herein, the term “diagnosed” means having been subjected to a physical examination by a person of skill, for example, a physician, and found to have a condition that can be diagnosed or treated by the compounds, compositions, or methods disclosed herein. For example, “diagnosed with cancer” means having been subjected to a physical examination by a person of skill, for example, a physician, and found to have a condition that can be diagnosed or treated by a compound or composition that can reduce tumor size or slow rate of tumor growth. A subject having cancer, tumor, or at least one cancer or tumor cell, may be identified using methods known in the art. For example, the anatomical position, gross size, and/or cellular composition of cancer cells or a tumor may be determined using contrast-enhanced MRI or CT. Additional methods for identifying cancer cells can include, but are not limited to, ultrasound, bone scan, surgical biopsy, and biological markers (e.g., serum protein levels and gene expression profiles). An imaging solution comprising a cell-sensitizing composition of the present invention may be used in combination with MRI or CT, for example, to identify cancer cells.

As used herein, “differentially expressed,” refers to the differential production of RNA, including but not limited to mRNA, tRNA, miRNA, siRNA, snRNA, and piRNA transcribed from a gene or regulatory region of a genome or the protein product encoded by a gene as compared to the level of production of RNA or protein by the same gene or regulator region in a normal or a control cell. In another context, “differentially expressed,” also refers to nucleotide sequences or proteins in a cell or tissue which have different temporal and/or spatial expression profiles as compared to a normal or control cell.

As used herein, “effective amount” refers to the amount of a compound provided herein that is sufficient to effect beneficial or desired biological, emotional, medical, or clinical response of a cell, tissue, system, animal, or human. An effective amount can be administered in one or more administrations, applications, or dosages. The term cam also include within its scope amounts effective to enhance or restore to substantially normal physiological function. The “effective amount” can refer to the amount of a reprogrammed neutrophil as described herein that can be effective to enhance anti-tumor immune responses.

As used herein, “gene” can refer to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long non-coding RNA and shRNA.

As used interchangeably herein, “subject,” “individual,” or “patient” can refer to a vertebrate organism, such as a mammal (e.g., human). “Subject” can also refer to a cell, a population of cells, a tissue, an organ, or an organism, preferably to human and constituents thereof.

As used herein, “pharmaceutical formulation” or “pharmaceutical composition” refers to the combination of an active agent, compound, or ingredient with a pharmaceutically acceptable carrier or excipient, making the composition suitable for diagnostic, therapeutic, or preventive use in vitro, in vivo, or ex vivo.

As used herein, “pharmaceutically acceptable carrier or excipient” refers to a carrier or excipient that is useful in preparing a pharmaceutical formulation that is generally safe, nontoxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

As used herein, “pharmaceutically acceptable salt” refers to any acid or base addition salt whose counter-ions are non-toxic to the subject to which they are administered in pharmaceutical doses of the salts.

As used herein, “preventative,” “prevent,” or “preventing” refers to hindering or stopping a disease or condition before it occurs, even if undiagnosed, or while the disease or condition is still in the sub-clinical phase.

As used interchangeably herein, the terms “sufficient” and “effective,” can refer to an amount (e.g., mass, volume, dosage, concentration, and/or time period) needed to achieve one or more desired result(s). For example, a therapeutically effective amount refers to an amount needed to achieve one or more therapeutic effects.

As used herein, “therapeutic” can refer to treating, healing, and/or ameliorating a disease, disorder, condition, or side effect, or to decreasing in the rate of advancement of a disease, disorder, condition, or side effect. A “therapeutically effective amount” can therefore refer to an amount of a compound that can yield a therapeutic effect. The therapeutic effect can be treating and/or preventing non-resolving cancer and/or related diseases or conditions.

As used herein, the term “treatment” or “treating” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease.

The term “weight percent,” “wt. %,” “wt-%,” “percent by weight,” “% by weight,” and variations thereof, as used herein, refer to the concentration of a substance as the weight of that substance divided by the total weight of the composition and multiplied by 100.

The present disclosure describes methods of reprogramming innate neutrophils into an immune-enhancing state conducive for the treatment of cancer. In some embodiments, the reprogrammed neutrophils described herein can be used to treat and/or prevent cancer in a subject in need thereof. In further embodiments, the reprogrammed neutrophils may reduce the tumor burden in the subject in need thereof.

In some aspects, neutrophil-based therapies hold significant advantages over traditional cancer therapies or emerging T-cell based immune therapies. First, neutrophils are the first natural responder to any abnormal situations including inflammation and cancer growth and has the innate ability to home into tumor tissues. Therefore, in some aspects, neutrophil-based therapies may eliminate key caveats associated with traditional bio-based therapies, including challenges in effectively targeting tumor tissues. In some embodiments, neutrophils may be used as an effective vehicle to deliver anti-cancer drugs to solid tumors. Second, neutrophils homed into cancer tissues may readily communicate with ever-evolving immune cells (e.g. T cells, NK cells, etc.) present in the tumor tissues. If properly programmed, tumor-homing neutrophils may naturally expand and/or activate existing tumor-associated immune-cells within the changing tumor immune environment, avoiding the limitation associated with CAR-T therapies which require constant engineering of T cells targeting evolving tumor antigens.

In some aspects, innate leukocytes such as neutrophils, are highly enriched in solid tumor tissues, adopting a pro-tumor phenotype. In these aspects, neutrophils promote tumor growth by serving as suppressors of adaptive immune cells through immune-suppressive mediators including CD11b, PD-L1 and ROS, as well as assisting tumor cell growth and metastasis through CD11b-mediated swarming/aggregation with cancer cells. In other aspects, neutrophils are also responsible for enhancing the broad-spectrum eradication of heterogeneous cancer cells that evade CAR-T therapies. Consistent with emerging single cell sequencing analyses, in some embodiments, at least three distinct subsets of neutrophils with varying levels of CD177 and CD11b are present. In some aspects, the CD177^{lo} population have a reduced expression of immune-suppressive markers such as CD11b, and preferentially express immune-enhancing mediators including, but not limited to, CD80, CD86, CD40, Dectin2 and EHD1. Therefore, in embodiments, the methods as described herein aim to expand the CD177^{lo} population for use in the treatment of cancer.

In some embodiments, a method of reprogramming neutrophils into immune-enhancing neutrophils are provided. In embodiments, the method comprises receiving innate neutrophils isolated from a heterologous or allogenic (homologous) donor. In some aspects, the innate neutrophils are isolated from human blood, including, for example, human peripheral blood. In further aspects, the neutrophils may be derived from stem cell culture. In embodiments, the innate neutrophils may be purified and optionally supplemented or treated with low dose granulocyte-macrophage colony-stimulating factor (GM-CSF) or low dose granulocyte colony-stimulating factor (G-CSF). In some aspects, the concentration of GM-CSF or G-CSF may be present in an amount of between about 0.1 ng/mL to about 5 ng/mL, between about 0.2 ng/mL to about 4.5 ng/mL, between about 0.5 ng/mL to about 4.0 ng/mL, between about 0.7 ng/mL to about 3 ng/mL, or between about 0.8 ng/mL to about 2 ng/mL. In some aspects, the GM-CSF may be used to maintain cell viability during the reprogramming process.

In some embodiments, the innate neutrophils are primed with a low-dose endotoxin to result in immune-enhancing neutrophils. In some aspects, low-dose (or “super low-dose endotoxin”) preferentially expands the CD177^{lo} immune-enhancing population. In examples, dosages in the “super low-dose” range will be in the picogram/milliliter (pg/mL) range. For example, between about 0.001 pg/mL and about 1000 pg/mL, between about 1 pg/mL and about 1000 pg/mL, or between about 10 pg/mL and about 1000 pg/mL. Without being limited to any particular theory of mechanism, the CD177^{lo} immune-enhancing neutrophils provide for potent immune-enhancing potential of reprogrammed neutrophils in reducing tumorigenesis. The immune-enhancing neutrophils may be isolated for further therapeutic uses as further described herein. While any method of isolation may be suitable, in some aspects, the immune-enhancing neutrophils may be isolated by flow cytometry or magnetic bead-based separation. In some embodiments, the low-dose endotoxin comprises lipopolysaccharide (LPS).

The term “endotoxin,” “low-dose endotoxin,” “super low-dose endotoxin,” and “lipopolysaccharide” (LPS) refer to bioactive compounds produced, in general, by Gram-negative bacteria, and constituting a major component of the bacterial outer membrane from which they may be released biologically or chemically. Endotoxins are amphiphilic molecules consisting of a lipid component, termed Lipid A, and a covalently bound polysaccharide. Because of genetic, biosynthetic, biological, and structural characteristics, the carbohydrate portion can be further divided into a Lipid A-proximal core region and a 0-specific side chain. It is understood in accordance with the disclosure that the endotoxin employed may be of any Gram-negative, endotoxin-carrying bacterium. In some embodiments, the endotoxin may be derived from Escherichia coli. In general, 0-specific chains are heteropolymers, made up of repeating oligosaccharide units (in enterobacteria up to 50) which consist of between two and eight monomers. The core region of LPS contains a complex oligosaccharide and, as regards to its structure, shows less variability in comparison to the 0-specific chain. In enterobacteria and some other families, one can differentiate between an outer core region with predominantly pyranosidic hexoses, such as D-glucose, D-galactose, 2-amino-2-deoxy-D-glucose or 2-amino-2-deoxy-D-galactose, and an inner core region. In all gram-negative bacteria, the 4 latter contains 3-deoxy-D-manno-oct-2-ulosonic acid (2-keto-3-deoxy-D-manno-octonic acid, Kdo) and often L-glycero-D-manno-heptose (Hep). Structurally, the Lipid A component forms the most uniform part of LPS. It can be separated from the carbohydrate portion by mild acid hydrolysis leading to the cleavage of the glycosidic bond between Kdo and Lipid A, and, hence, becomes accessible to a detailed structural elucidation. The term LPS comprises S-form and R-form LPS and substructures such as Lipid A and partial structures thereof.

In embodiments, low-dose endotoxin and super low-dose endotoxin preferentially induces a low-grade inflammatory response from monocytes, while a higher dose of endotoxin drastically causes tolerance and exhaustion of monocytes. Therefore, in some aspects, the concentration of endotoxin for priming the innate neutrophils are provided in a range of between about 25 pg/mL and about 150 pg/mL, between about 35 pg/mL and about 140 pg/mL, between about 45 pg/mL and about 130 pg/mL, between about 55 pg/mL and about 120 pg/mL, between about 65 pg/mL and about 120 pg/mL, between about 75 pg/mL and about 110 pg/mL, between about 85 pg/mL and about 110 pg/mL, and between about 90 pg/mL and about 110 pg/mL. As would be appreciated by those skilled in the art, immune-enhancing neutrophils may be isolated without the use of a low-dose endotoxin as a primer.

In some embodiments, the low-dose endotoxin is applied to the innate-enhancing neutrophils for a period of at least 1 hour, at least 2 hours, at least 3 hours, or at least 4 hours.

In embodiments, the reprogrammed neutrophils express immune-enhancing surface markers. In some aspects, the immune-enhancing neutrophils express surface markers comprising CD177^{lo} and at least one of Dectin2 (Clec4n)^{hi} or EHD1^{hi}. In embodiments, the surface markers comprise CD177^{lo} and Dectin 2 (Clec4n)^{hi}. In other embodiments, the surface markers comprise CD177^{lo} and EHD1^{hi}. In even further embodiments, the surface markers may comprise CD177^{lo}, Dectin2 (Clec4n)^{hi}, and EHD1^{hi}. In some aspects, the immune-enhancing neutrophils comprising the surface markers identified herein exhibit elevated expression of immune-enhancing genes comprising CD44, CD80, CD86, CD74, EHD1, Dectin2, CD40, CD200R, CD62L, or a combination thereof. In further aspects, the immune-enhancing neutrophils may further exhibit a reduced expression of immune-suppressive markers, such as CD11b. The elevated expression of immune-enhancing genes paired with a reduced expression of immune-suppressive markers provide for compositions having efficacy as a treatment for cancer.

In some examples, the low-dose endotoxin preferentially reprograms neutrophils into an immune-enhancing state, conducive for the treatment of a disease, such as cancer. Single cell RNA sequencing (scRNAseq) of neutrophils trained by super-low dose endotoxin can identify the reprogramming of the immune-enhancing neutrophil cluster after treatment with the endotoxin. In implementations, the transfusion of low-dose endotoxin-trained neutrophils (or the immune-enhancing neutrophils) can effectively reduce the tumor-burden in a subject in need thereof. Low-dose endotoxin-trained neutrophils may also relieve the suppression of adaptive T cells as compared to naïve neutrophils.

Without being limited to any particular mechanism or theory, the low-dose endotoxin can selectively clear away or remove the innate suppressor IRAK-M and initiate a sustained activation of STAT5 and reprogramming of the neutrophils into an immune-enhanced state. In some aspects, the genetic deletion of IRAK-M may result in an enhanced anti-tumor immune environment in reducing tumorigenesis in cancers. The suppression of IRAK-M may involve the activation of immune-proteasome which can be activated during the initial response of innate leukocytes to challenges. In some embodiments, the priming of the innate neutrophils with the low-dose endotoxin may result in a reduction of IRAK-M in the immune-enhancing neutrophils.

Therefore, the disclosures further provide for methods of treating cancer in a subject, wherein the method comprises the steps in reprogramming neutrophils into immune-enhancing neutrophils as described herein. In aspects, the method further comprises a step of administering the immune-enhancing neutrophils to a subject in need thereof. In embodiments, the immune-enhancing neutrophils expressing surface markers comprising CD177^{lo} and at least one of Dectin2 (Clec4n)^{hi} or EHD1^{hi} are isolated prior to administering to the subject in need thereof. In some embodiments, the subject is a mammal. In further embodiments, the mammal is a human.

In some embodiments, the immune-enhancing neutrophils may be provided as a pharmaceutical formulation, or pharmaceutical composition, that can include a pharmaceutically acceptable carrier thereof. The pharmaceutical compositions can be used to treat and/or prevent diseases, such as cancer. The immune-enhancing neutrophils may be provided in a dosage form. The dosage forms can be adapted for administration by any appropriate route. In embodiments, the preferred route of administration is via intravenous injection. In further embodiments, other appropriate routes can include, but are not limited to, epidural, intracranial, intraocular, vaginal, intraurethral, parenteral, intracranial, subcutaneous, intramuscular, intravenous, intraperitoneal, intradermal, intraosseous, intracardiac, intraarticular, intracavernous, intrathecal, intravitreal, intracerebral, gingival, subgingival, intracerebroventricular, and intradermal.

Dosage forms adapted for parenteral administration and/or adapted for any type of injection (e.g., intravenous, intraperitoneal, subcutaneous, intramuscular, intradermal, intraosseous, epidural, intracardiac, intraarticular, intracavernous, gingival, subginigival, intrathecal, intravireal, intracerebral, and intracerebroventricular) can include aqueous and/or non-aqueous sterile injection solutions, which can contain anti-oxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and resuspended in a sterile carrier to reconstitute the dose prior to administration. Extemporaneous injection solutions and suspensions can be prepared in some embodiments, from concentrated cell solutions, sterile powders, granules, and tablets.

For some embodiments, the dosage form contains a predetermined amount of the immune-enhanced neutrophils per unit dose. In some embodiments, the predetermined amount of the immune-enhancing neutrophils is a therapeutically effective amount of the immune-enhancing neutrophils effective to treat or prevent cancer. In other embodiments, the predetermined amount of the immune-enhancing neutrophils can be an appropriate fraction of the therapeutically effective amount of the active ingredient (e.g., the immune-enhancing neutrophils and/or auxiliary active agent). Such pharmaceutical formulations may be prepared by any of the methods well known in the art. In some embodiments, the immune-enhancing neutrophils are administered to the subject in the form of a blood transfusion via an intravenous dose of administration. The amount of blood containing the immune-enhancing neutrophils to be transferred via the blood transfusion may be in the range of between about 50 mL and about 500 mL, between about 55 mL and about 400 mL, between about 60 mL and about 390 mL, between about 70 mL and about 380 mL, between about 80 mL and about 370 mL, between about 90 mL and about 360 mL, or between about 90 mL and about 350 mL.

In embodiments, the immune-enhancing neutrophils may be administered to the subject at least once weekly, at least two times per week, or at least three times per week. In some embodiments, the immune-enhancing neutrophils may be administered to the subject once or more than once per day. In other embodiments, the immune-enhancing neutrophils and pharmaceutical formulations thereof can be administered one or more times per month, such as 1 to 5 times per month, such as 1, 2, 3, 4 or 5 times per month. In still further embodiments, the immune-enhancing neutrophils and pharmaceutical formulations thereof can be administered one or more times per year, such as 1 to 11 times per year. In some embodiments, the chemically programmed neutrophils and pharmaceutical formulations thereof can be administered once daily, once weekly, at least once per month, at least twice per month, at least three times per month, or at least four times per month.

In some embodiments, the methods and compositions of the disclosure may be used to treat cancer. In aspects, the cancer may include, but is not limited to, bladder cancer, head and neck cancer, pancreatic ductal adenocarcinoma (PDA), pancreatic cancer, colon carcinoma, mammary carcinoma, breast cancer, fibrosarcoma, mesothelioma, renal cell carcinoma, lung carcinoma, thyoma, prostate cancer, colorectal cancer, ovarian cancer, acute myeloid leukemia, thymus cancer, brain cancer, squamous cell cancer, skin cancer, eye cancer, retinoblastoma, melanoma, intraocular melanoma, oral cavity and oropharyngeal cancers, gastric cancer, stomach cancer, cervical cancer, head, neck, renal cancer, kidney cancer, liver cancer, ovarian cancer, prostate cancer, colorectal cancer, esophageal cancer, testicular cancer, gynecological cancer, thyroid cancer, acquired immune deficiency syndrome (AIDS)-related cancers (e.g., lymphoma and Kaposi's sarcoma), viral-induced cancers such as cervical carcinoma (human papillomavirus), B-cell lymphoproliferative disease and nasopharyngeal carcinoma (Epstein-Barr virus), Kaposi's Sarcoma and primary effusion lymphomas (Kaposi's sarcoma herpesvirus), hepatocellular carcinoma (hepatitis B and hepatitis C viruses), and T-cell leukemias (human T-cell leukemia virus-1), glioblastoma, esophogeal tumors, hematological neoplasms, non-small-cell lung cancer, chronic myelocytic leukemia, diffuse large B-cell lymphoma, esophagus tumor, follicle center lymphoma, head and neck tumor, hepatitis C virus infection, hepatocellular carcinoma, Hodgkin's disease, metastatic colon cancer, multiple myeloma, non-Hodgkin's lymphoma, indolent non-Hodgkin's lymphoma, ovary tumor, pancreas tumor, renal cell carcinoma, small-cell lung cancer, or stage IV melanoma.

In some embodiments, the disclosure relates to a method of treating a solid tumor cancer. In aspects, the solid tumor cancer is selected from bladder cancer, non-small cell lung cancer, cervical cancer, anal cancer, pancreatic cancer, squamous cell carcinoma including head and neck cancer, renal cell carcinoma, melanoma, ovarian cancer, small cell lung cancer, glioblastoma, gastrointestinal stromal tumor, breast cancer, lung cancer, colorectal cancer, thyroid cancer, bone sarcoma, stomach cancer, oral cavity cancer, oropharyngeal cancer, gastric cancer, kidney cancer, liver cancer, prostate cancer, colorectal cancer, esophageal cancer, testicular cancer, gynecological cancer, thyroid cancer, colon cancer, and brain cancer. In some embodiments, the cancer is at least one of colon, lung, breast, bladder, or prostate cancer.

In optional embodiments, the immune-enhancing neutrophils and pharmaceutical formulations thereof may be co-administered with a secondary agent by any convenient route. The secondary agent is a separate compound and/or pharmaceutical formulation from the immune-enhancing neutrophils or pharmaceutical formulations thereof. The secondary agent can be administered simultaneously with the immune-enhancing neutrophils or pharmaceutical formulations thereof. The secondary agent can be administered sequentially with the immune-enhancing neutrophils or pharmaceutical formulations thereof. Suitable secondary agents include, but are not limited to, DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, and chemotherapeutics.

All publications and patent applications in this specification are indicative of the level of ordinary skill in the art to which this disclosure pertains. All publications and patent applications are herein incorporated by reference to the same extent as if each individual publication or patent application was specifically and individually indicated as incorporated by reference.

### EXAMPLES

Embodiments of the present disclosure are further defined in the following non-limiting Examples. It should be understood that these Examples, while indicating certain embodiments of the disclosure, are given by way of illustration only. From the above discussion and these Examples, one skilled in the art can ascertain the essential characteristics of this disclosure, and without departing from the spirit and scope thereof, can make various changes and modifications of the embodiments of the disclosure to adapt it to various usages and conditions. Thus, various modifications of the embodiments of the disclosure, in addition to those shown and described herein, will be apparent to those skilled in the art from the foregoing description. Such modifications are also intended to fall within the scope of the appended claims.

### Example 1

### Materials and Methods

Data source: The single cell RNA sequencing (scRNAseq) data sets were deposited into the NCBI Genebank with an accession number of GSE230237.

Mice: Wild type (WT) C57BL/6 mice were originally from Jackson laboratory and bred and maintained in the animal facility at Virginia Tech in accordance to approved Animal Care and Use Committee protocol. All littermate mice were 8-10 weeks of age and 25-30 g weight when experiments were initiated.

Experimental Design: WT mice received a single intraperitoneal injection of azoxymethane (AOM, Sigma-Aldrich) at a dose of 10 mg/kg body weight. A week after AOM injection, the mice were given three cycles of 2% dextran sulfate sodium salt (DSS, MP Biomedicals) for 5 days followed by 14 days of normal drinking water. To prime neutrophils in vitro, bone marrow neutrophils from WT mice were purified (>90% confirmed by flow cytometry) using EasySep™ Mouse Neutrophil Enrichment Kit (Stem Cell), according to the manufacturer's instructions. Purified neutrophils were cultured in with RPMI completed medium (10% fetal bovine serum, 2 mM L-glutamine, 10 mM HEPES, 1% penicillin/streptomycin) supplemented with 1 ng/ml GM-CSF and treated with LPS (100 pg/ml) or PBS as control overnight. Then the neutrophils were harvested and resuspended in PBS. Recipient WT mice were transfused twice (post DSS day 5 and day 12) per DSS-resting cycle through intravenous injection with 2.5×10⁶ WT neutrophils. After the last water cycle mice were sacrificed and tissues were harvested for further analysis. Body weight, stool consistency, and bleeding were measured as part of a clinical score (score 0-4, with higher score corresponding to worse condition). Depending on the size, polyp formation was classified as macro-polyp (equal to or greater than 2 mm) and micro-polyp (less than 2 mm). Independent experiments of AOM-DSS induced colorectal tumorigenesis were conducted more than 3 times, and for each experiment there were at least 5 mice in each group.

Single Cell RNAseq, Cell Clustering and Analyses: Cultured neutrophils were used for single cell sequencing and analyses. Ly6G+ positive neutrophils were purified from bone marrow via flow cytometry-based selection, and cultured with either PBS, or 100 pg/ml LPS overnight in completed RPMI medium supplemented with 1 ng/ml GM-CSF. Cultured cells were processed to prepare libraries using the 10× Genomics Chromium Single Cell 3′ v3 Kit. Following 12 cycles of amplification, the qualities of cDNA samples were checked via Bioanalyzer and sequenced on Illumina® HiSeq platform, with the paired-end read length of 150 bp at each end plus 8-bp i7 index. scRNAseq data were analyzed by Seurat (version 3.1.2) in R. The default settings of Seurat were used to perform quality control, normalization, and scaling of the data. Cells with more than 20% of reads from mitochondrial genes, and cells that have more than 6,500 or fewer than 200 unique genes were removed. Data were normalized and scaled before dimensionality reduction which was performed by principal component analysis (PCA), and UMAP were used for cell clustering. Marker genes that were differentially expressed in at least 10% of cells within a target cluster were obtained by using the non-parametric Wilcoxon rank sum test in R.

Lamina Propria Cell Isolation: Colons were opened longitudinally and cleaned by flushing with ice-cold PBS. Single-cell suspension was prepared using Lamina Propria dissociation Kit (MACS). The colons were cut into ˜5 mm long pieces, and incubated with HBSS containing 5 mM EDTA, 5% FBS, and 1 mM DTT to remove epithelial cells. The remaining pieces were then cut into fine pieces and incubated with HBSS containing 5% FBS and enzyme mix using the gentleMACS™ dissociator. The cells were washed, passed through a 70 um strainer, and resuspended in FACS buffer for further flow cytometry analyses.

Flow Cytometry: Fluorescent-conjugated anti-mouse antibodies specific for CD80, CD40, OX40, CD44, Cd74, EHD1, Dectin2, Dectin1, CD11b, CD4, CD8, PD-L1, CD62L, Ly6G, Ki67, PD-1, Tim3, granzyme B, IFNγ, CD107a, CD122, CD200R, CD69, NKG2A, Foxp3 were purchased from BioLegend®. Propidium iodide (PI) was also added to determine the cell viability. Leukocytes from bone marrow, spleen, and mesenteric lymph nodes were harvested as previously described. The single cells were stained with fluorescently-labelled antibodies in the presence of Fc block in FACS buffer (1×HBSS supplemented with 2% FBS and 0.05% sodium azide) for 20 min on ice. Cytosol intracellular staining was performed using Cytofix/Cytoperm™ Plus fixation/permeabilization solution kit (BD Biosciences). Intracellular staining of Foxp3 and Ki67 was performed using Fixation/Permeabilization Solution Kit (ebioscience). Stained cells were analyzed with a FACSCanto II (BD Biosciences). FACS plots were analyzed with FlowJo.

T Cell Proliferation and Activation Assay: Splenic T cells were purified using EasySep™ Mouse T Cell Isolation Kit (Stem Cell), according to the manufacturer's instructions. Purified T cells were labelled with 5,6-carboxyfluorescein diacetate succinimidyl (CFSE) (Invitrogen, Molecular Probe), according to the manufacturer's instructions. CFSE-labeled T cells were mixed with primed neutrophils at a 1:1 ratio and co-cultured in anti-CD3 antibody (2 μg/ml) coated plates in the presence of anti-CD28 antibody (2 μg/ml) for 72 hours. CFSE signals and activation markers were analyzed by flow cytometry on gated CD4+ and CD8+ cells.

For human neutrophils, peripheral blood samples from healthy donors were purchased from Research blood components. Neutrophils were isolated by using MojoSort™ whole blood human neutrophil isolation kit (BioLegend®). Human neutrophils were cultured in RPMI 1640 completed medium supplemented with human GM-CSF (1 ng/ml) and treated with LPS (100 pg/ml) or PBS as control for 24 hours. For human T cells, peripheral blood was mixed with PBS (2% FBS) at 1:1 ratio and carefully loaded onto Ficoll® Paque Plus (Sigma), then centrifuged for 30 min at 800 g. Peripheral blood mononuclear cells were collected and subjected to MojoSort™ human T cell isolation kit (BioLegend®). CFSE-labeled T cells were mixed with primed neutrophils at a 1:1 ratio and co-cultured in anti-CD3 antibody (1 μg/ml) coated plates in the presence of anti-CD28 antibody (1 μg/ml) for 72 hours. CFSE signals were analyzed by flow cytometry.

NK Cell Proliferation Assay: NK cells were purified from splenocytes using EasySep™ Mouse NK Cell Isolation Kit (Stem Cell), according to the manufacturer's instructions. CFSE-labeled NK cells were mixed with primed neutrophils at a 1:2 ratio and co-cultured in RPMI completed medium supplemented with IL-2 (25 ng/ml) for 4 days. CFSE signals as well as NKG2A expression were analyzed by flow cytometry.

NK Cell Killing Assay: YAC-1 cells were purchased from ATCC. NK cells were purified from splenocytes and cultured in RPMI completed medium supplemented with IL-2 (25 ng/ml) for 4 days. After wash, NK cells were mixed with neutrophils at a 1:1 ratio. After a 2-hour incubation period in RPMI completed medium, CFSE-labeled YAC-1 were added in the co-culture system for additional 4 hours. Live fluorescent-labeled YAC-1 cells were counted by addition of Bright Absolute Counting Beads (Life Technologies) via flow cytometry. Lactate dehydrogenase (LDH) release in conditioned medium were measured by using colorimetric Lactate Dehydrogenase Assay kit (Abcam).

Immunoblotting: Primed neutrophils were lysed in SDS lysis buffer (1% SDS, 10% glycerol, 0.05M Tris-HCl pH6.8) with phosphatase and proteinase inhibitor cocktails (Sigma). Protein lysis was applied to SDS-PAGE and transferred to a polyvinylidene difluoride membrane. The membrane was blocked and probed with anti-IRAK-M (Abcam), anti-pSTAT5 (Cell Signaling) anti-STAT5 (Cell Signaling), anti-PSMD10 (Cell Signaling), anti-KLF4 (Abcam) or anti-3-actin (Santa Cruz) antibody, followed by horseradish peroxidase-linked secondary antibodies (Cell Signaling) and chemiluminescence ECL detection kit (Thermo Scientific). Chemiluminescence signals were detected using the Fujifilm Intelligent Dark Box. Immunoblots were quantified using ImageJ.

ELISA: The levels of IFNγ and granzyme B in colon lysate and conditioned medium were measured using ELISA kits purchased from R&D system, according to the manufacturer's instructions. To prepare tissue lysate, colons were cut into small pieces and lyzed in T-PER™ tissue protein extraction reagent (ThermoFisher®) by sonication. Cytokines in the colon were normalized by total protein weight.

Statistical Analysis: All experiments were performed at least 3 times. Representative and reproducible results were shown. Statistical analysis was performed with Prism GraphPad Software 10.0. Values were expressed as means±SD. The significance of the differences was assessed by Student's t-test (for two groups) or one-way ANOVA (for multiple groups). P<0.05 was considered statistically significant.

### Example 2

### Reprogramming of Immune-Enhancing Neutrophils by Super-Low Dose Endotoxin

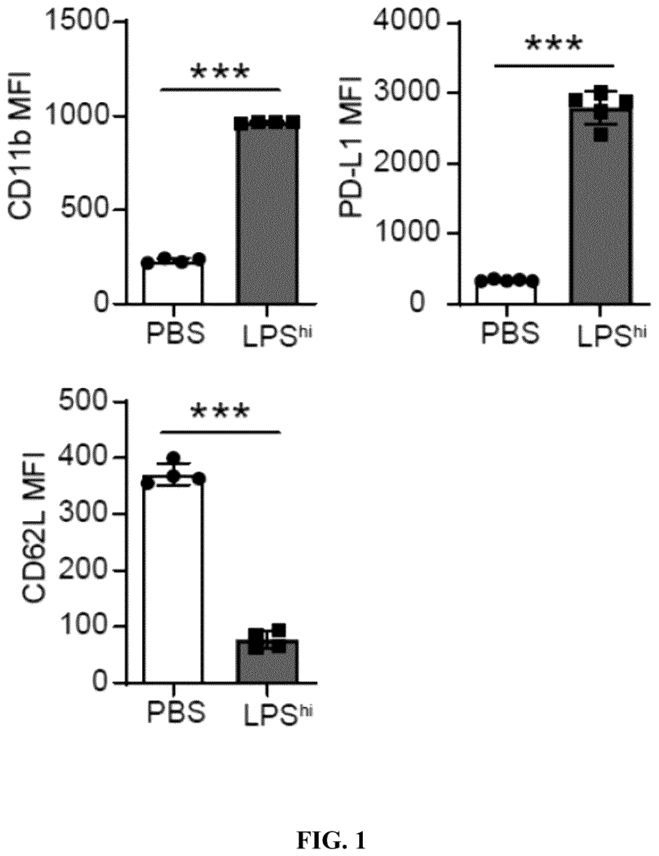

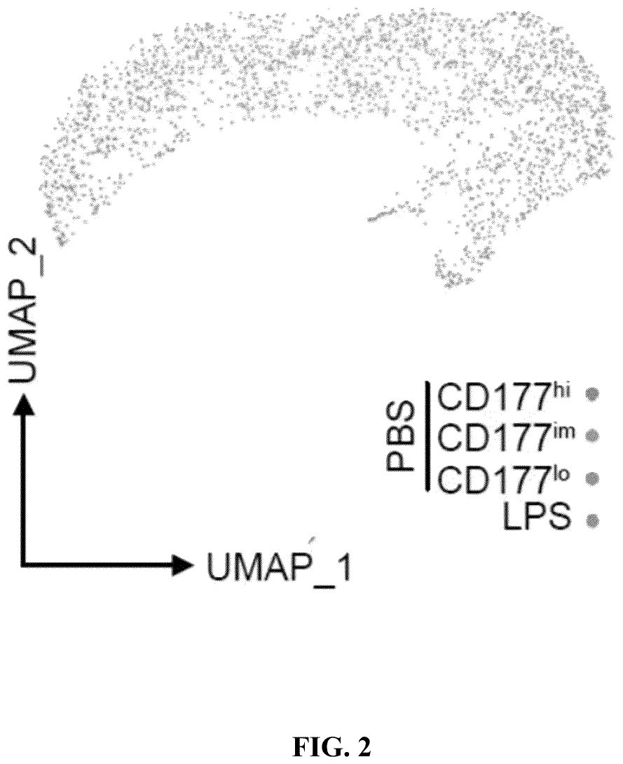

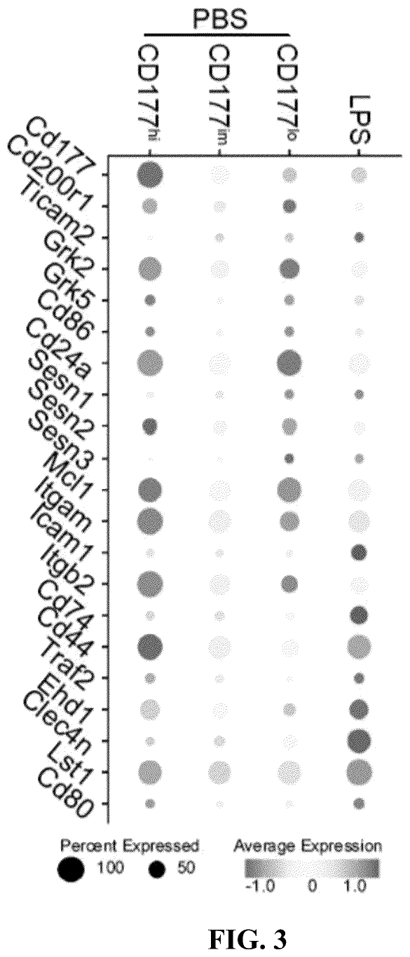

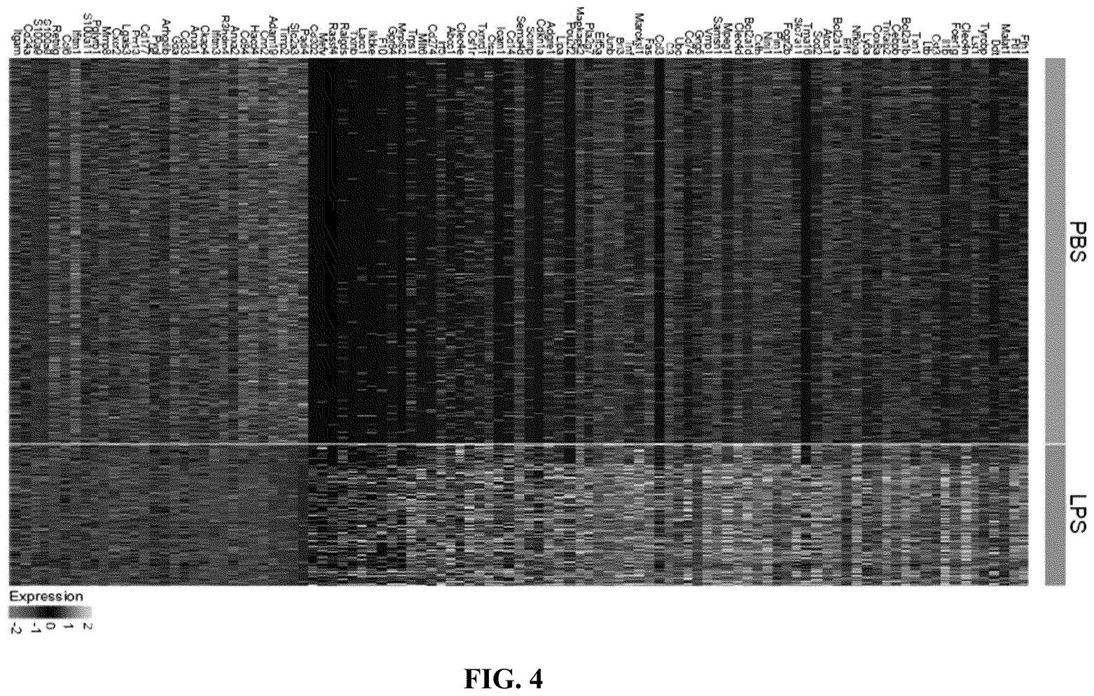

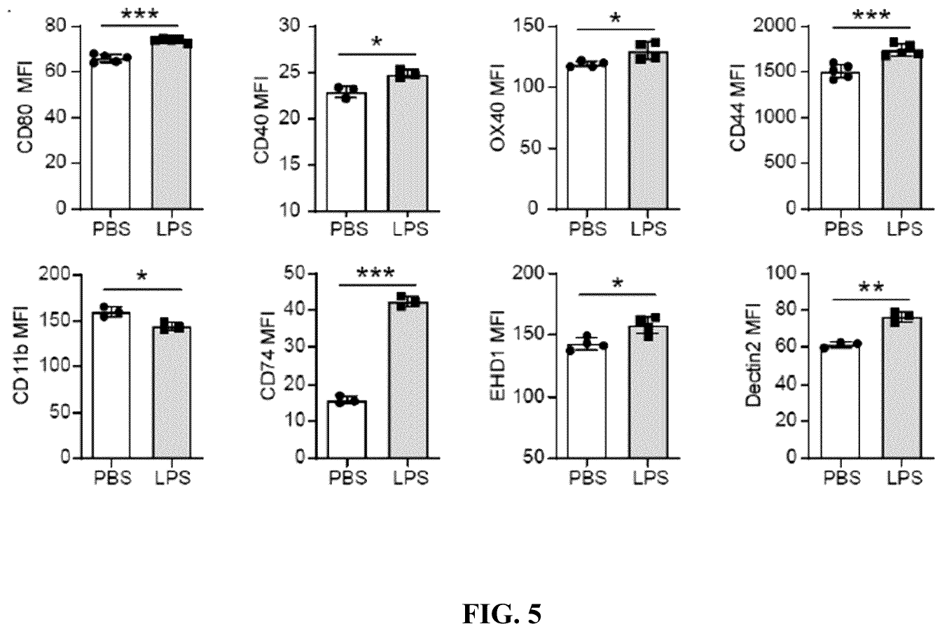

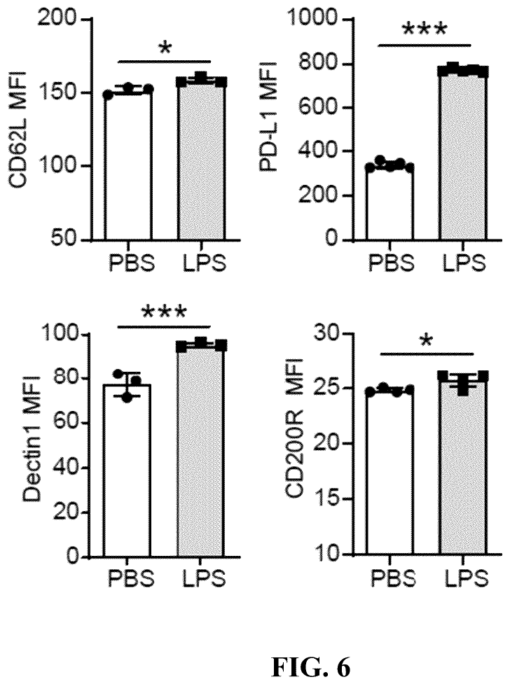

The effects of reprogramming immune-enhancing neutrophils were evaluated utilizing the methods as described in Example 1. The data revealed that LPS could dose-dependently modulate monocyte activation status, with prolonged high dose LPS causing immune suppression, and prolonged subclinical super-low dose LPS generating immune-enhancement. In terms of neutrophils, the effects of high dose LPS have been examined with elevated expression of immune-suppressive molecules such as PD-L1 and CD11b (FIG. 1). However, the effect of super-low dose LPS has not been well-characterized. Within this example, single cell analyses were performed of murine bone marrow neutrophils challenged with super-low dose LPS (100 pg/ml) in the presence of GM-CSF to maintain cell viability within the 24 hr treatment period. As shown in FIG. 2, naïve neutrophils cluster into three clusters, CD177hi, CD177intermediate, and CD177lo/CD200Rhi. Upon overnight treatment with super-low dose LPS, neutrophils were projected into a unique CD177lo cluster, characterized by reduced expression of immune-suppressive CD11b and elevated expression of immune-enhancing genes including CD44, CD80, CD74, EHD1, Dectin2, and CD40 (FIGS. 3 and 4). This surprising result was further validated at the protein level via flow cytometry. As shown in FIG. 5 and FIG. 6, neutrophils programmed by subclinical super-low dose LPS exhibited elevated expression of CD44, CD80, CD40, EHD1, Dectin2, CD200R, CD62L, and reduced expression of CD11b. This is in sharp contrast with neutrophils treated with higher dose LPS causing immune-suppression, with elevated levels of CD11b and shredding of CD62L (FIG. 1).

### Example 3

### Reprogrammed Neutrophils by Low-Dose Endotoxin Enhance Host Anti-Tumor Defense

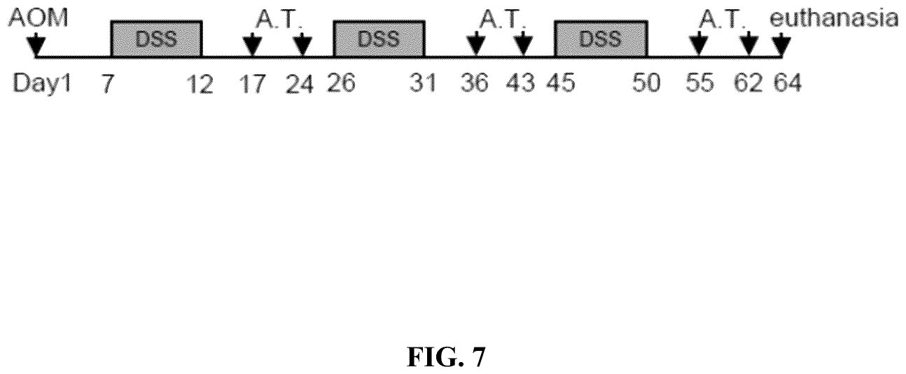

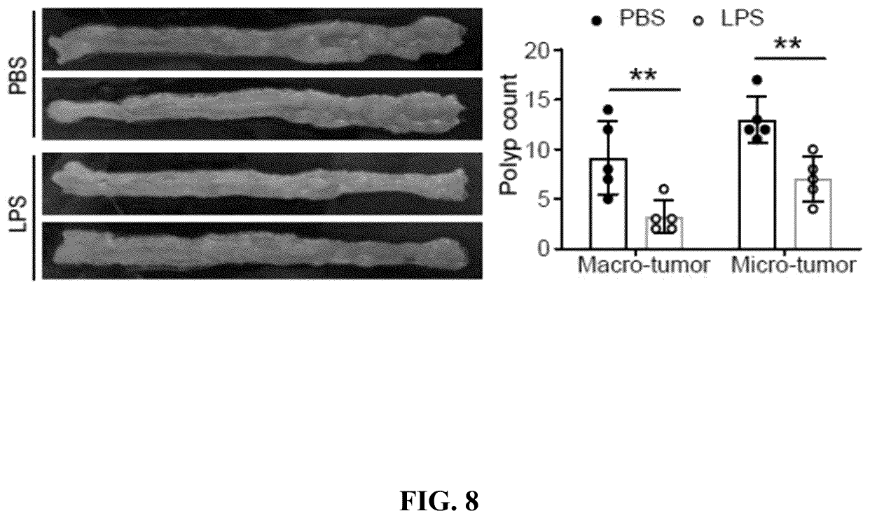

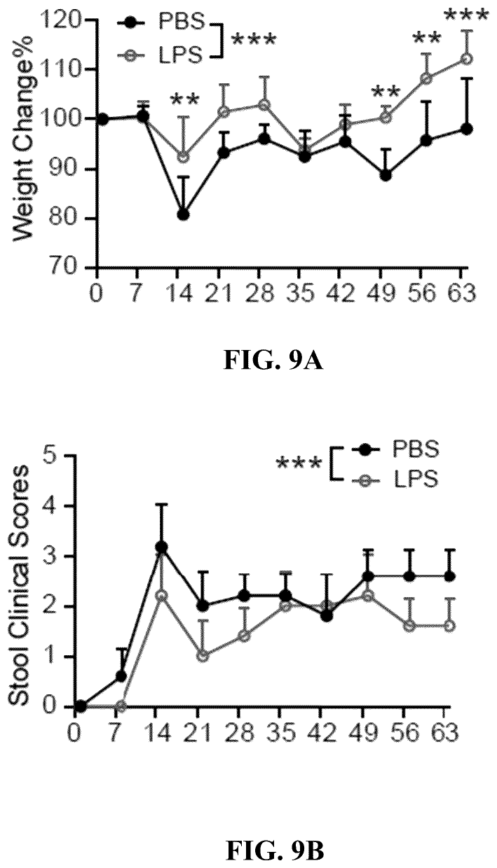

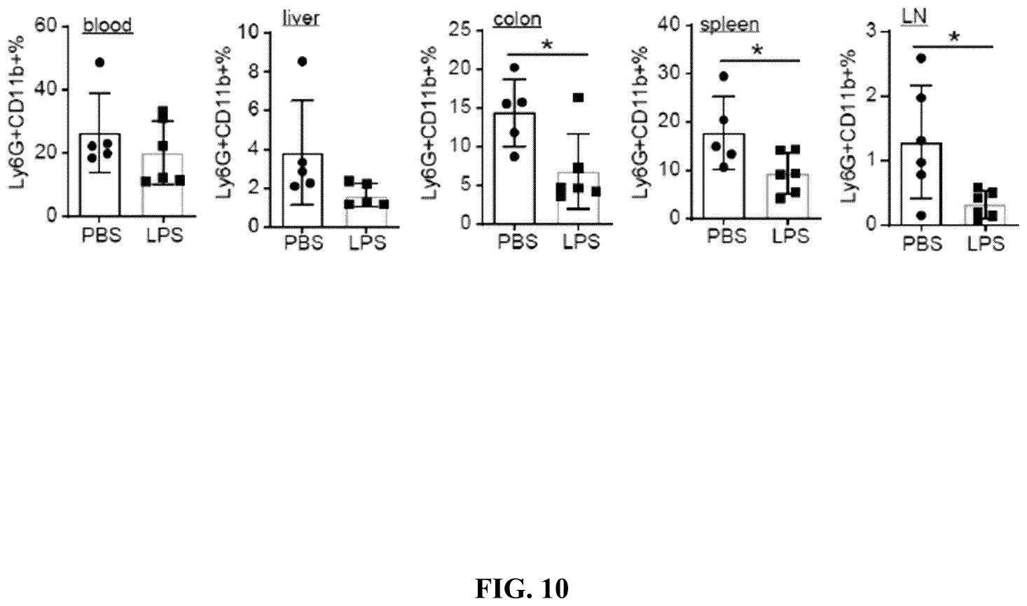

The scRNAseq analyses with super-low dose LPS hearkens back to the decade-old concept of “Coley's toxin” where Dr. Coley experimented with injecting a tiny amount of microbial extracts into patients with cancer. The present example evaluated whether neutrophils precisely trained with a subclinical dose of LPS in vitro could be used to treat cancer in experimental animals. Azoxymethane (AOM)-dextran sulfate sodium salt (DSS) regimen was used, which is a well-defined colorectal cancer model. First, bone marrow neutrophils were isolated and primed with LPS (100 pg/ml) or PBS as control. LPS or PBS-primed neutrophils were then given via an intraperitoneal injection (i.p.) weekly to WT mice subjected to AOM-DSS challenge as described in the Method section (FIG. 7). As shown in FIG. 8, the control group developed significant amount of colon tumors after AOM-DSS treatment. The mice receiving LPS-primed neutrophils exhibited a significant reduction in the tumor loads. In particular, the average numbers of microscopic (<2 mm diameter) and macroscopic (≥2 mm diameter) polyps in the mice receiving LPS-primed neutrophils were reduced by 50-65% compared with that in the mice receiving PBS-primed neutrophils. Moreover, the mice receiving LPS-primed neutrophils displayed reduced weight loss and much lower disease scores (FIG. 9A and FIG. 9B) during the course of AOM-DSS treatment. In addition, consistent with less severity of colonic symptoms at the end of AOM-DSS regimen, lower percentages of infiltrating neutrophils in the colon, liver, spleen and mesenteric lymph nodes from LPS-primed neutrophil recipients were observed (FIG. 10). The data demonstrated that the transfusion of LPS-primed neutrophils enhanced anti-tumor activities against AOM-DSS induced colon tumorigenesis.

### Example 4

### Reprogrammed Neutrophils by Low-Dose Endotoxin Enhance Anti-Tumor Immune Environment In Vivo

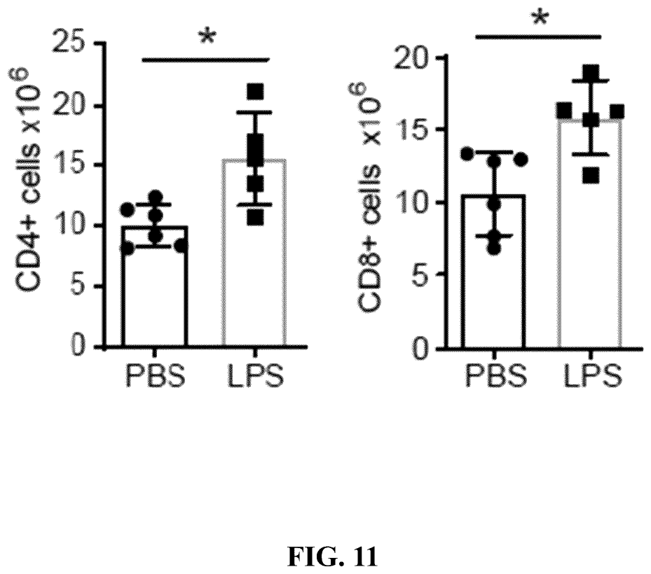

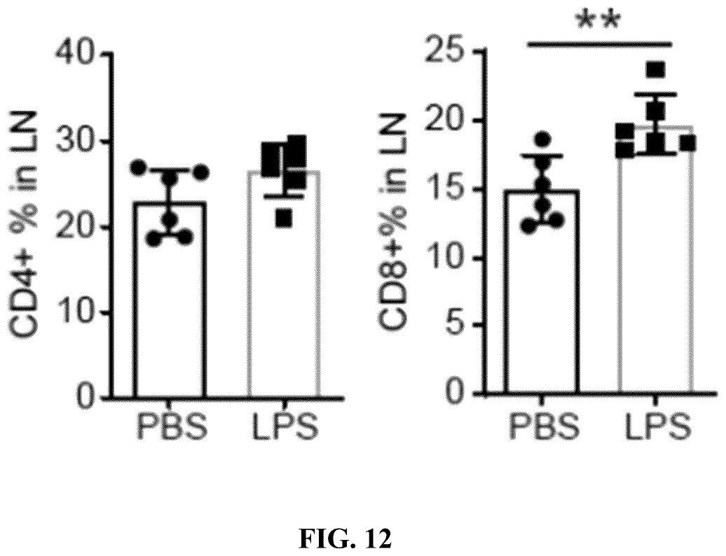

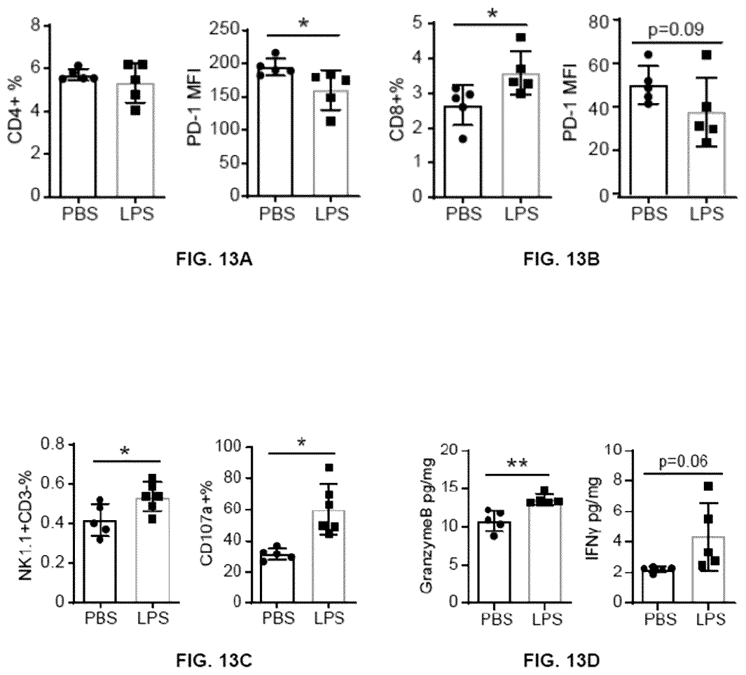

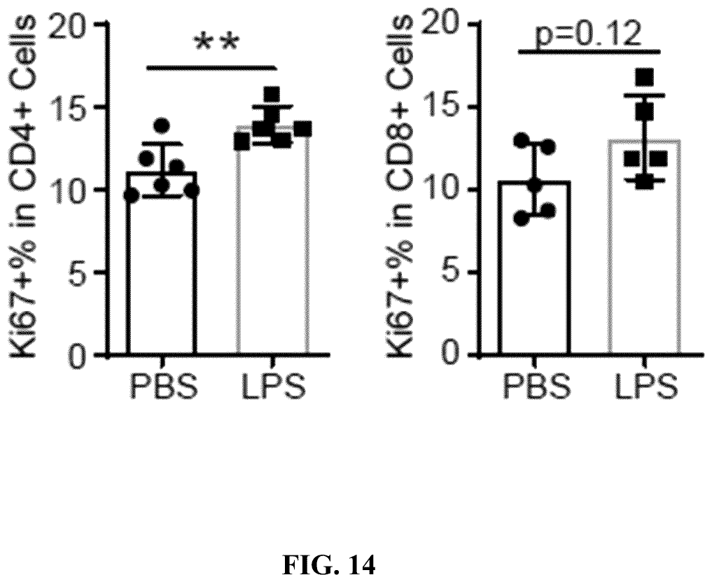

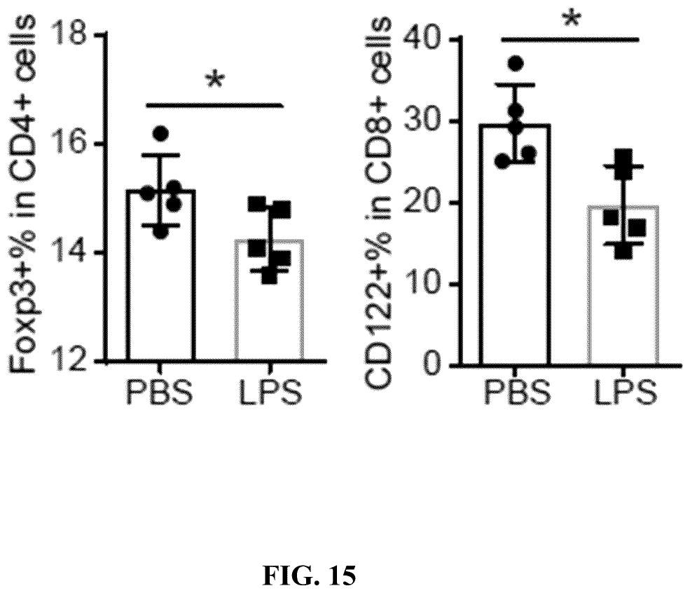

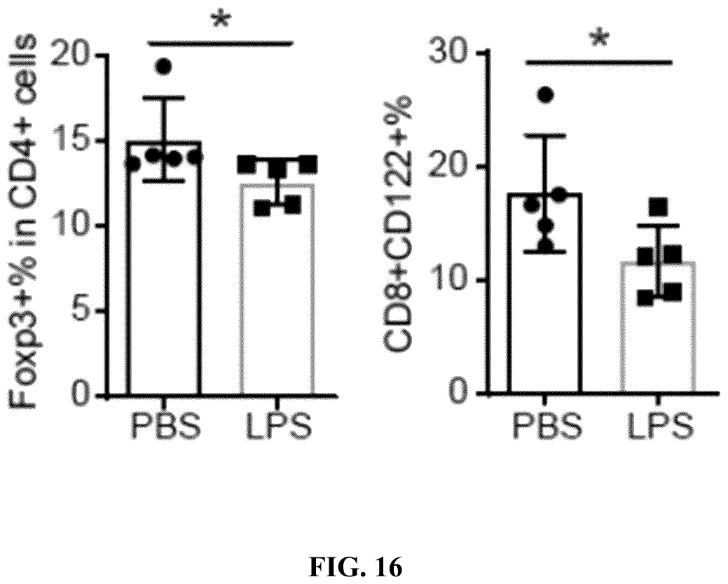

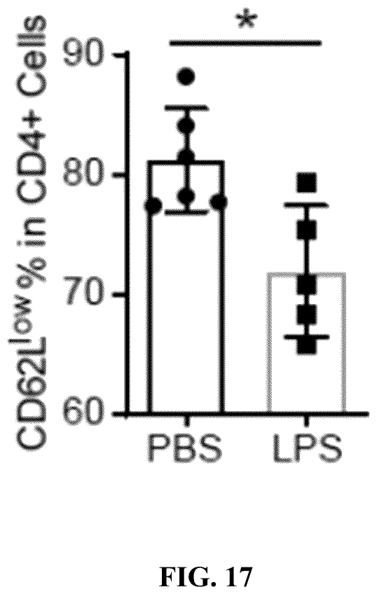

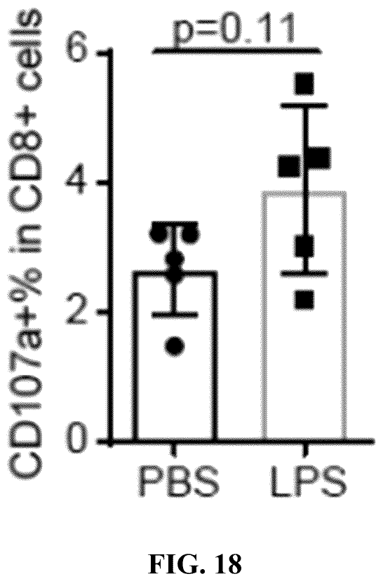

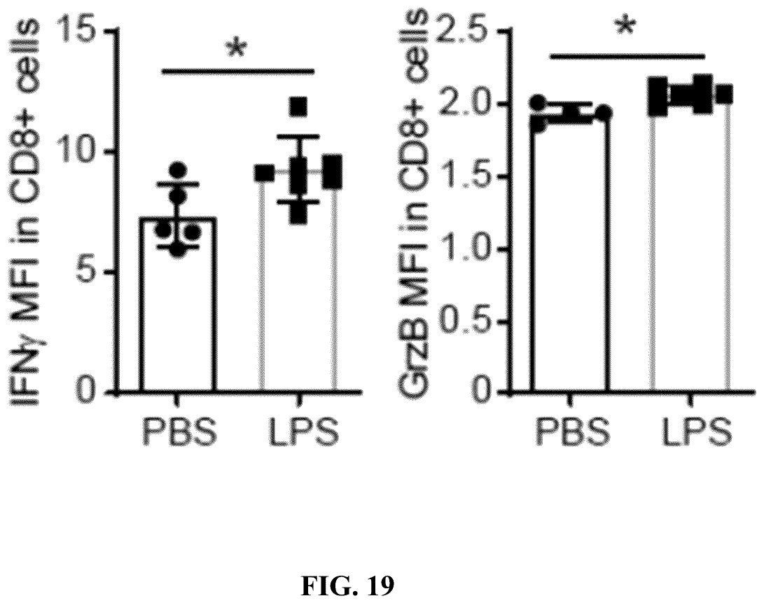

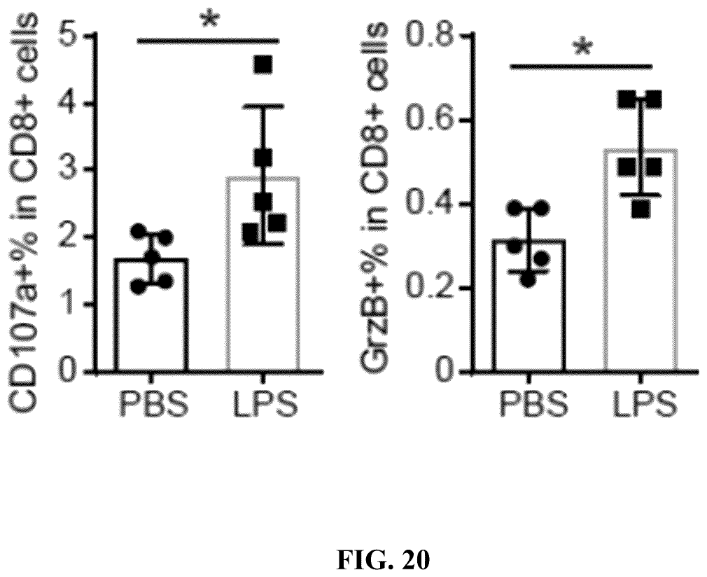

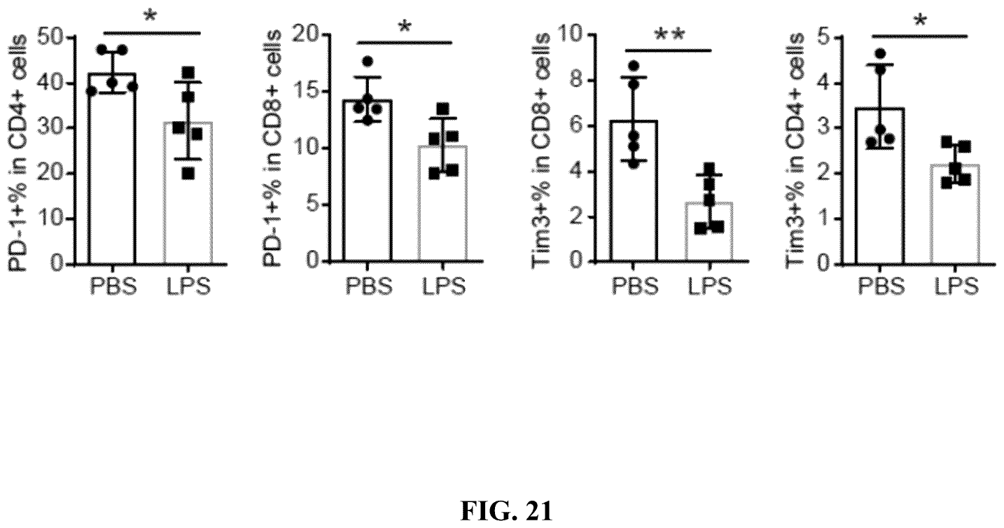

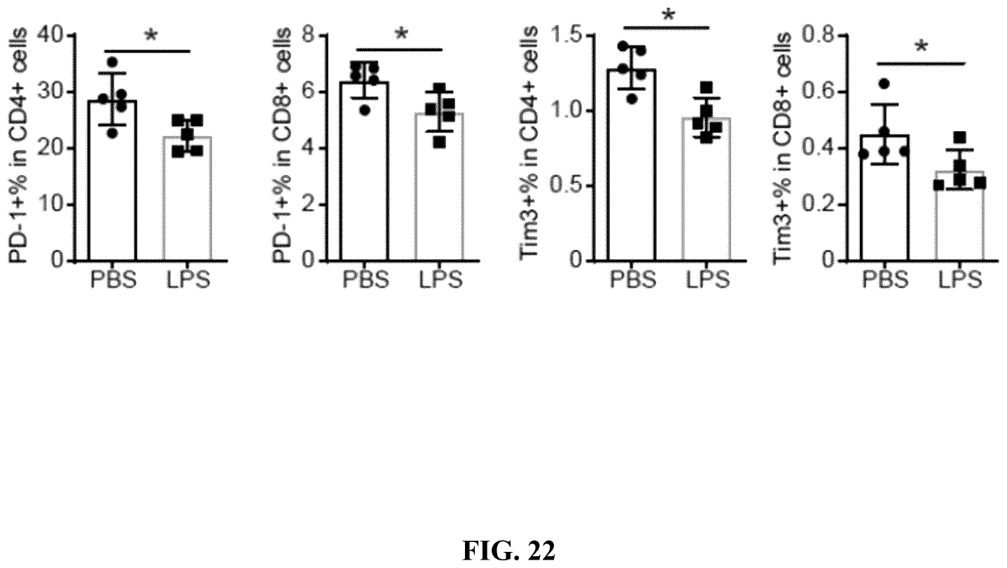

The T cell status after transfusion of LPS-primed neutrophils were further evaluated utilizing the methods described in Example 1. Significantly higher cell counts of CD4 positive and CD8 positive T cells were observed in the spleen (FIG. 11), mesenteric lymph nodes (FIG. 12), and colon (FIGS. 13A, 13B, 13C, and 13D) of the mice receiving LPS-primed neutrophils compared with the mice receiving PBS-primed neutrophils during AOM-DSS treatment. The percentage of Ki67+ CD4 T cells was much higher in the group of LPS-primed neutrophil recipients (FIG. 14). Moreover, the reduction of Foxp3+ CD4 Treg cells and CD122+ CD8 Treg cells were observed in both spleen and lymph modes from the group of LPS-primed neutrophil recipients compared with the control group (FIG. 15 and FIG. 16). Furthermore, not only cell number but also activation status of both CD4 and CD8 T cells were induced by weekly transfusion of LPS-primed neutrophils. In particular, the shredding of CD62L in CD4 T cells from LPS-primed neutrophil recipients were significantly increased (FIG. 17). The percentage of CD107a+ CD8 T cells were much higher in LPS-primed neutrophil recipients, and the expression of granzyme B and interferon 7 in CD8 T cells were significantly increased as well (FIG. 18, FIG. 19, and FIG. 20). On the other hand, the expression of PD-1 and Tim3, immune checkpoint molecules, were remarkably decreased in the CD4 and CD8 T cells in the spleen and lymph nodes from LPS-primed neutrophil recipients compared with the control group (FIG. 21 and FIG. 22).

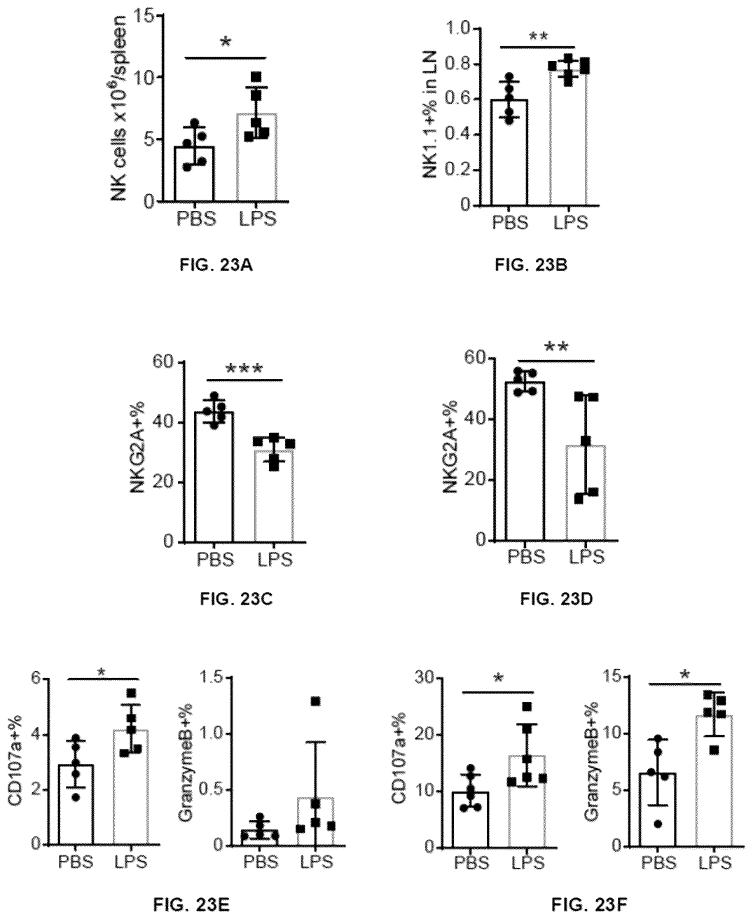

NK cells also play an essential role in anti-tumor immunity, therefore, whether the transfusion of LPS-primed neutrophils affected NK cells in vivo was further evaluated. First, the cell counts of NK cells were significantly and persistently higher in the spleen (FIG. 23A) and mesenteric lymph nodes (FIG. 23B) from the mice receiving LPS-primed neutrophils. Moreover, the expression of NKG2A, an immune check point, was also down-regulated on the NK cells from the mice receiving LPS-primed neutrophils (FIGS. 23C and 23D). Meanwhile, NK cell cytokine production in LPS-primed neutrophil recipients was enhanced, as reflected with higher level of CD107a on NK cells, as well as a higher percentage of granzyme B positive cells in the mice receiving LPS-primed neutrophils (FIGS. 23E and 23F). It was further observed that the percentages of colonic NK cells from mice receiving LPS-primed neutrophils were higher than those from control mice after AOM/DSS treatment (FIG. 13C), and CD107a expression level was also remarkably upregulated.

Consistent with elevated activated T cells and NK cells, enhanced anti-tumor environments were detected in the colon, supported by the significant increase of granzyme B and interferon 7 in the colon of LPS-primed neutrophil recipients (FIG. 13D).

### Example 5

### Reprogrammed Neutrophils by Low-Dose Endotoxin Relieve Adaptive Immune Suppression In Vitro

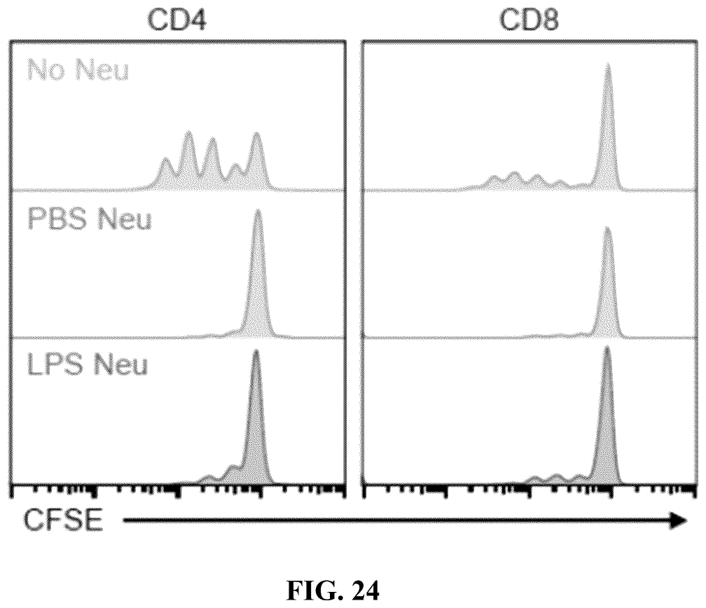

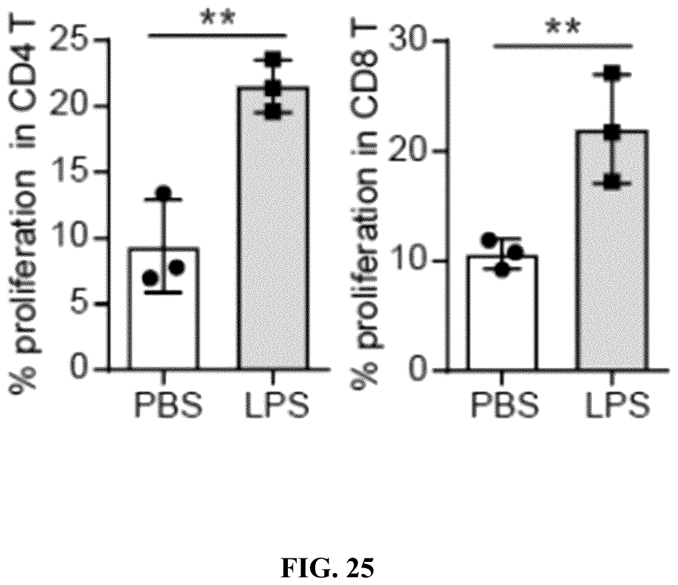

To further explore whether super-low dose of LPS priming could enhance T cell function, in vitro co-culture assays were further conducted according to the methods provided in Example 1. The primed neutrophils were mixed with CFSE-labeled allogeneic T cells in a CD3-coated plate for 72 hours. PBS-treated neutrophils showed a typical immunosuppressive phenotype as evidenced from reduced T cell proliferation with the addition of neutrophils (FIG. 24 and FIG. 25). However, when co-cultured with LPS-treated neutrophils, the proliferation of CD4 and CD8 T cells were markedly augmented. The similar enhance effects on T cell proliferation by LPS-primed neutrophils were also observed in the syngeneic murine T cell and neutrophil co-culture system, and human T cell and neutrophil coculture system (FIG. 24 and FIG. 25).

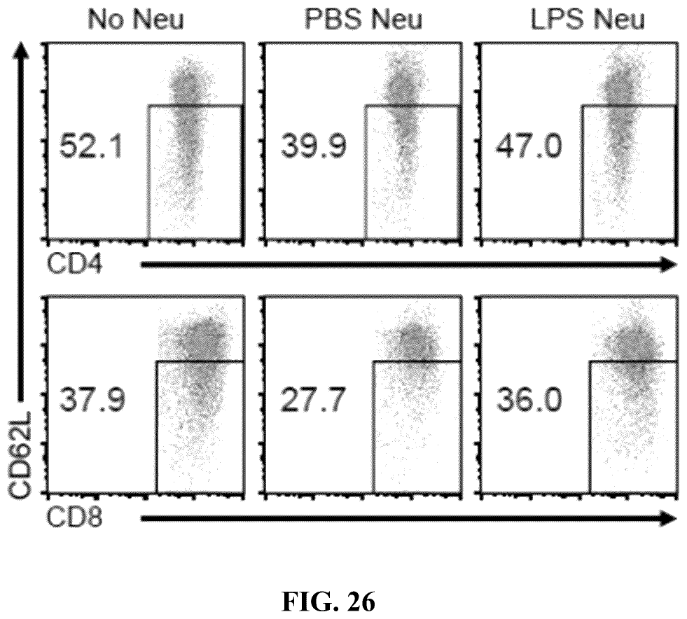

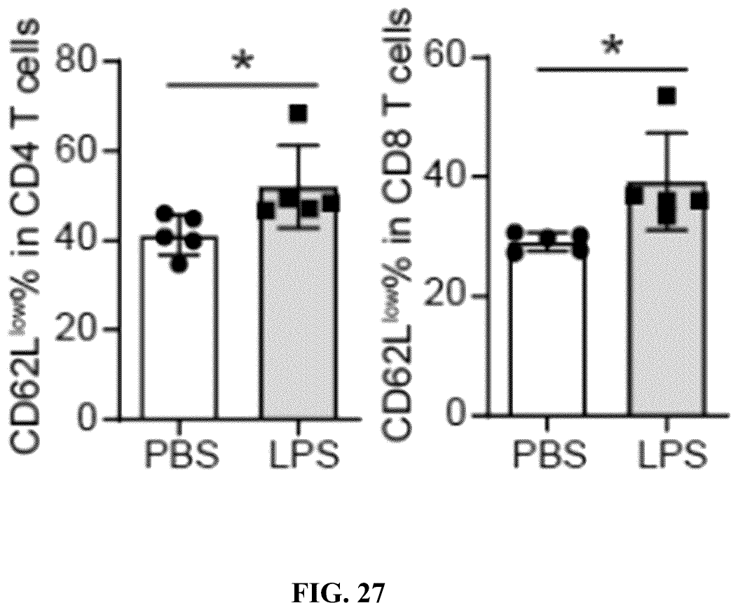

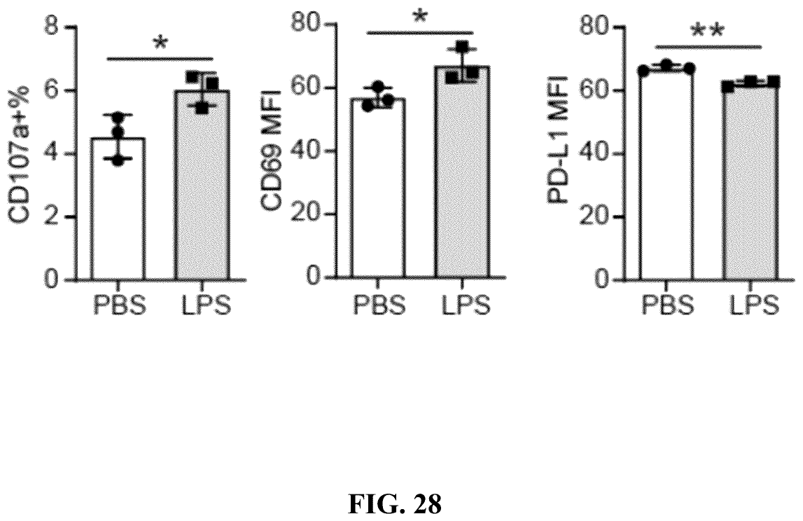

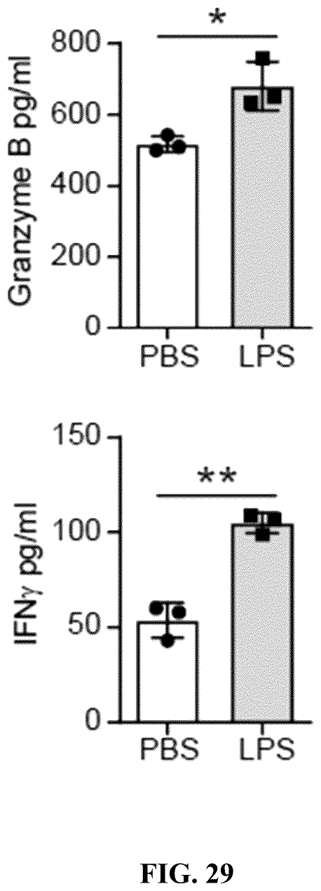

Further, the effect of neutrophils on T cell activation and cytokine production were further observed. Down-regulation of CD62L were observed in the T cells co-cultured with LPS-primed neutrophils, compared with the T cells co-cultured with PBS-primed neutrophils (FIG. 26 and FIG. 27). Moreover, when co-cultured with LPS-primed neutrophils, CD8 T cells exhibited significant elevated activation status, supported by the higher level of CD69 and CD107a as well as reduced PD-1 expression (FIG. 28). Additionally, the concentrations of Granzyme B and IFNγ were increased in the co-culture with LPS-primed neutrophils (FIG. 29). These data further confirm that T cell activation was enhanced when co-cultured with LPS-primed neutrophils as compared to PBS-primed neutrophils.

### Example 6

### Reprogrammed Neutrophils Enhance NK Cell Activity In Vitro

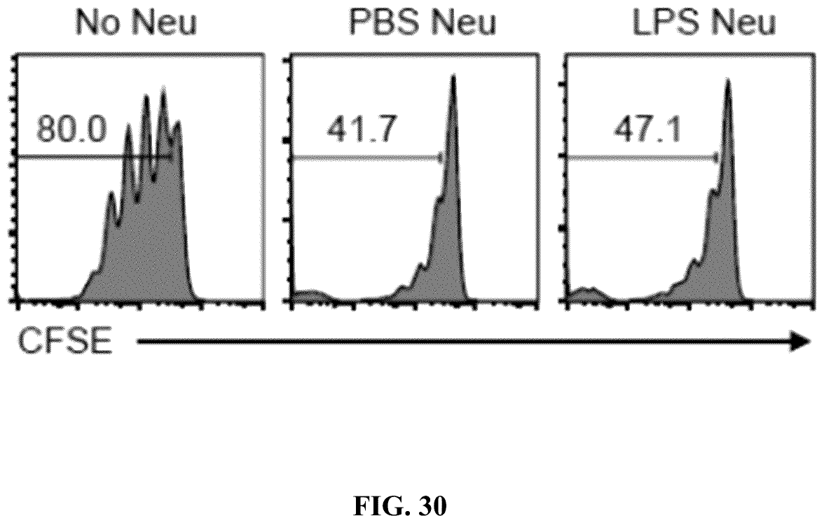

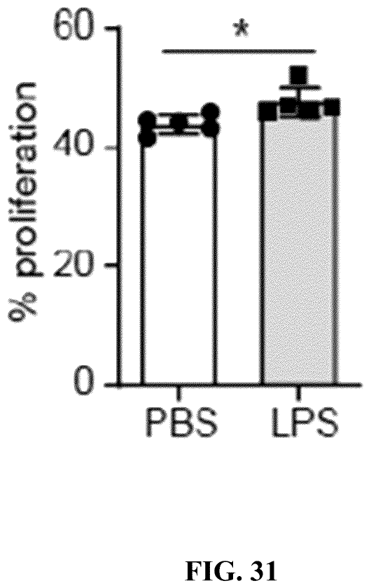

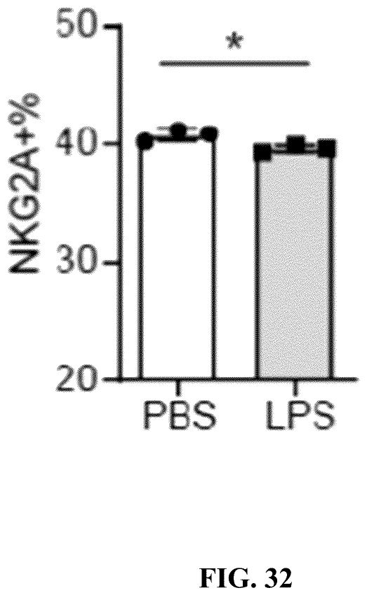

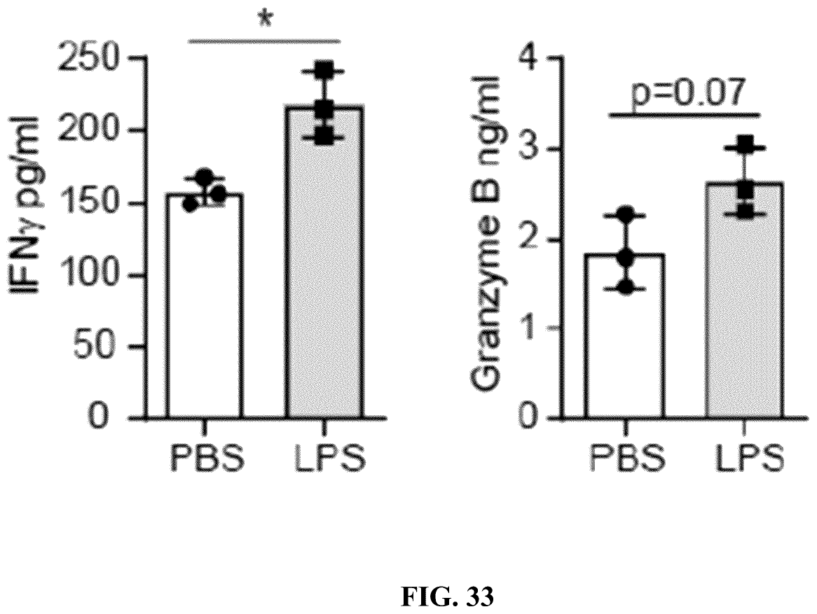

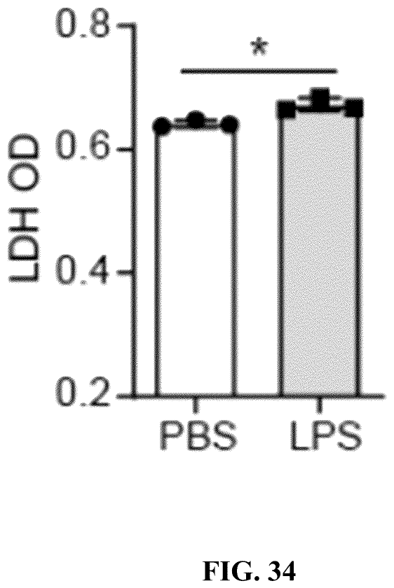

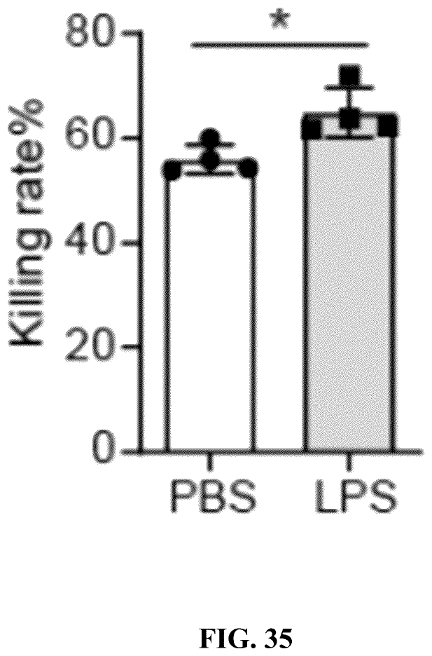

As described and demonstrated in previous examples, not only did T cells interact with neutrophils, however, NK cells further interacted with neutrophils (FIGS. 23A, 23B, 23C, and 23D). Within the present example, splenic NK cells were isolated and co-cultured with either PBS- or LPS-primed neutrophils in the presence of IL2. The proliferation of NK cells was suppressed by the addition of neutrophils. However, compared with PBS-primed neutrophils, LPS-primed neutrophils partially released the suppression of NK cell proliferation (FIG. 30 and FIG. 31). NKG2A is known as an immune checkpoint of NK cells. The percentage of NKG2Ahi NK cells was significantly reduced when NK cells were co-cultured with LPS-primed neutrophils (FIG. 32). Next, the function of NK cells in the presence of neutrophils was tested. The secretion of granzyme B and IFNγ were elevated when NK cells were co-cultured with LPS-primed neutrophils as compared to PBS-primed neutrophils (FIG. 33). The killing activities of NK cells to target YAC-1 cells were evaluated by both colorimetric measurement of LDH level in the culture medium and flow cytometry. Higher levels of LDH were detected in the medium with LPS-primed neutrophils than the controls (FIG. 34). Higher killing rates also were observed by counting the live fluorescent labeled YAC-1 cells via flow cytometry (FIG. 35).

Collectively, these data suggest that subclinical super-low dose LPS enhanced both T cell and NK cell functions in vitro.

### Example 7

### Low-Dose Endotoxin Reprograms Neutrophils Through Activating STAT5 and Reducing IRAK-M

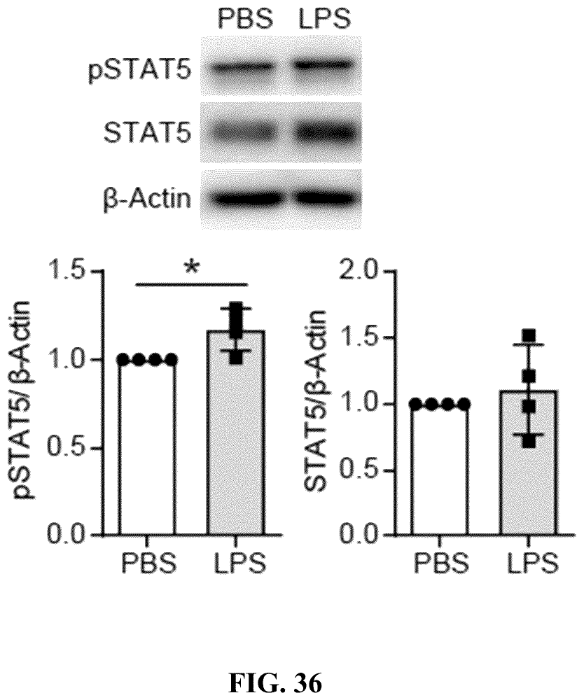

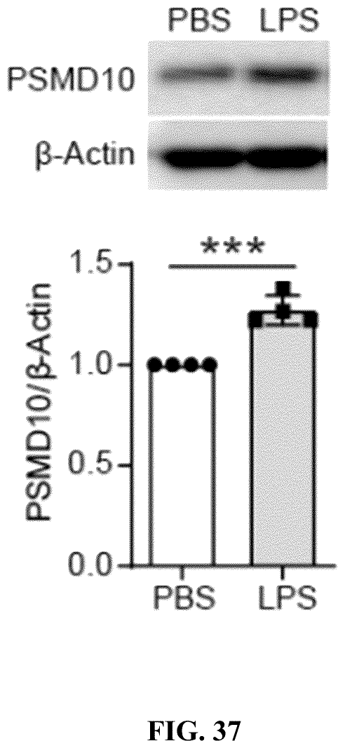

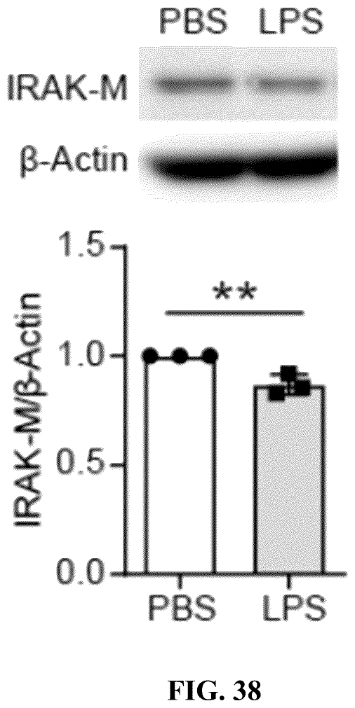

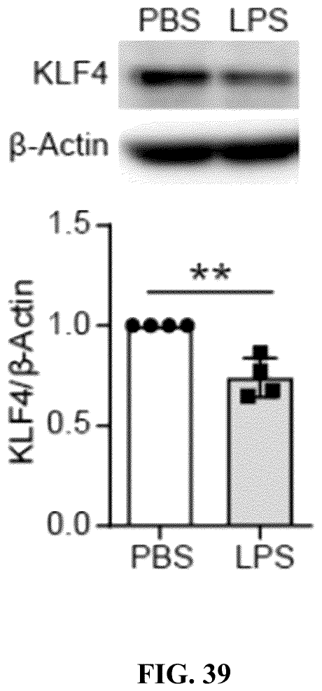

The molecular mechanisms responsible for the immune-enhancing effects of super-low dose LPS were examined. As shown with the monocyte studies, monocytes treated with super-low dose LPS initiated an activation of STAT5 as well as proteasome activation leading to the degradation of immune-suppressors such as IRAK-M. The present example evaluated whether super-low dose LPS may similarly activate the program of STAT5 and immune-proteasome in neutrophils. As shown in FIG. 36, neutrophils treated with super-low dose LPS exhibit elevated levels of phospho-STAT5, likely responsible for the sustained expression of immune-enhancing mediators. In the meantime, super-low dose LPS priming significantly elevated the levels of proteasome subunit PSMD10 (FIG. 37). The levels of IRAK-M, a known immune suppressor in innate leukocytes, was evaluated and a significant reduction of IRAK-M in LPS-primed neutrophils was observed (FIG. 38). IRAK-M not only suppressed the immune-enhancing signals, but was also shown to be involved in the activation of immune suppressors such as CD11b. We previously demonstrated that IRAK-M deficient neutrophils had significantly reduced expression of CD11b. Complementing the earlier observations, it was demonstrated that neutrophils treated with super-low dose LPS exhibited reduced levels of KLF4 (FIG. 39), a known transcription factor involved in CD11b expression. Collectively, the data suggest that the removal of IRAK-M related suppressors may enable the establishment of immune-enhancing neutrophils by super-low dose LPS.

The data within all of the Examples clarify the novel reprogramming potential of super-low dose endotoxin on neutrophils, shifting them from an immune-suppressive state to an immune-enhancing phenotype. The results clarify the mystique surrounding the original concept of “Coley's toxin” in experimenting with low-levels of bacterial extract for improving anti-tumor immune responses. While neutrophils exhibit both immune-suppressive and immune-enhancing potentials, the data reveal that the fate of neutrophils bifurcates depending upon the signal strength of endotoxin, with super-low dose endotoxin preferentially promoting the immune-enhancing polarization. This conclusion is not only supported by in vitro characterization of reprogrammed neutrophils, but also by in vivo proof of principle evidence showing enhanced anti-tumor defense in a recipient mice receiving the reprogrammed neutrophils with super-low dose endotoxin.

The data within the Examples identify the immune-enhancing property of super-low dose endotoxin. The results clarify a long-held uncertainty involving the early observation of “Coley's toxin” in treating cancer patients, in which the administration of bacterial extracts into human cancer patients led to variable clinical manifestations. Initial practices involved carefully dosing a tiny amount of bacterial extracts that was just enough to barely induce a systemic low-fever response from the patients. Due to the inevitable variations, the Coley's toxin was largely ignored in favor of more reproducible radiation- and chemo-therapies in the last decade. Even with the new dawn of immune-therapies and referral of “Coley's toxin” as the foundation for the emergence of immune-therapies, limited efforts were paid to evaluate the potential of innate leukocyte-based therapies. Instead, the mainstream of cancer-immune therapies has been related to adaptive immunity coupled with check-point inhibition. However, adaptive immune-based therapies suffer from the caveat of tumor evasion due to high mutation rates, similar to the drawbacks related to the vaccine approach targeting emerging pathogens. Revisiting innate-based cancer therapies may aid in generating effective and broad-spectrum anti-cancer therapies.

The above specification provides a description of the manufacture and use of the disclosed compositions and methods. Since many embodiments can be made without departing from the spirit and scope of the disclosure, the disclosure resides in the claims.

What is claimed is:

1. A method of reprogramming neutrophils into immune-enhancing neutrophils, comprising:
receiving innate neutrophils isolated from a heterologous or allogenic donor; and
priming the innate neutrophils with a low-dose endotoxin to result in immune-enhancing neutrophils,
wherein the immune-enhancing neutrophils express surface markers comprising CD177^{lo} and at least one of Dectin2 (Clec4n)^{hi} or EHD1^{hi}.

2. The method of claim 1, wherein the low-dose endotoxin comprises lipopolysaccharide (LPS).

3. The method of claim 1, wherein the low-dose endotoxin is provided at a concentration of between about 10 pg/mL and about 1000 pg/mL.

4. The method of claim 1, wherein the surface markers comprise CD177^{lo} and Dectin 2 (Clec4n)^{hi}, or wherein the surface markers comprise CD177^{lo} and EHD1^{hi}.

5. The method of claim 1, wherein the immune-enhancing neutrophils exhibit elevated expression of immune-enhancing genes comprising CD44, CD80, CD86, CD74, EHD1, Dectin2, CD40, CD200R, CD62L, or a combination thereof.

6. The method of claim 5, wherein the immune-enhancing neutrophils exhibit a reduced expression of CD11b.

7. The method of claim 1, wherein the priming of the innate neutrophils with the low-dose endotoxin result in a reduction of IRAK-M in the immune-enhancing neutrophils.

8. The method of claim 1, further comprising a step of isolating the immune-enhancing neutrophils expressing surface markers comprising CD177^{lo} and at least one of Dectin2 (Clec4n)^{hi} or EHD1^{hi} by flow cytometry or magnetic bead-based separation.

9. The method of claim 1, wherein the innate neutrophils are isolated from human peripheral blood, and wherein the innate neutrophils are further treated with low dose granulocyte-macrophage colony-stimulating factor (GM-CSF) or granulocyte colony-stimulating factor (G-CSF).

10. The method of claim 9, wherein the concentration of low dose GM-CSF or G-CSF is in a range of from about 0.1 ng/mL to about 5 ng/mL.

11. A method of treating cancer in a subject, the method comprising:
receiving innate neutrophils isolated from a heterologous or allogenic donor;
priming the innate neutrophils with a low-dose endotoxin to result in immune-enhancing neutrophils, wherein the immune-enhancing neutrophils express surface markers comprising CD177^{lo} and at least one of Dectin2 (Clec4n)^{hi} or EHD1^{hi}; and
administering the immune-enhancing neutrophils to the subject.

12. The method of claim 11, wherein the innate neutrophils are isolated from human peripheral blood, and wherein the innate neutrophils are further treated with low dose granulocyte-macrophage colony-stimulating factor (GM-CSF) or granulocyte colony-stimulating factor (G-CSF).

13. The method of claim 11, wherein the immune-enhancing neutrophils are administered to the subject at least once per month or at least once weekly.

14. The method of claim 11, wherein the immune-enhancing neutrophils are administered to the subject in the form of a blood transfusion via an intravenous route of administration.

15. The method of claim 11, wherein the low-dose endotoxin comprises lipopolysaccharide (LPS), and wherein the LPS is provided at a concentration of between about 10 pg/mL and about 1000 pg/mL.

16. The method of claim 11, wherein the surface markers comprise (i) CD177^{lo} and Dectin 2 (Clec4n)^{hi}; (ii) CD177^{lo} and EHD1^{hi}; or (iii) CD177^{lo}, Dectin2 (Clec4n)^{hi} and EHD1^{hi}.

17. The method of claim 11, wherein the immune-enhancing neutrophils exhibit elevated expression of immune-enhancing genes comprising CD44, CD80, CD86, CD74, EHD1, Dectin2, CD40, CD200R, CD62L, or a combination thereof, and wherein the immune-enhancing neutrophils exhibit a reduced expression of CD11b.

18. An anti-tumor agent for treating cancer comprising:
an immune-enhancing neutrophil, wherein the immune-enhancing neutrophil expresses surface markers comprising CD177^{lo} and at least one of Dectin2 (Clec4n)^{hi} or EHD1^{hi}; and
a pharmaceutically acceptable carrier.

19. The anti-tumor agent from claim 18, wherein the immune-enhancing neutrophil has been pre-treated with a low-dose endotoxin.

20. The anti-tumor agent from claim 19, wherein low-dose endotoxin comprises lipopolysaccharide.
